(19) United States
(12) Patent Application Publication
(10) Pub. No.: US 2008/0213768 A1
(43) Pub. Date: Sep. 4, 2008

(54) Identification and use of biomarkers for non-invasive and early detection of liver injury

(76) Inventors: Zhauhui Cai, Garnet Valley, PA (US); Marie Coffin, Cary, NC (US); Keith Allen, Cary, NC (US); Brian Bullard, Las Cruces, NM (US); Alan J. Higgins, Chapel Hill, NC (US)

Correspondence Address: HUTCHISON LAW GROUP PLLC, PO BOX 31686, RALEIGH, NC, 27612, US

(21) Appl. No.: 11/893,845

(22) Filed: Aug. 17, 2007

Related U.S. Application Data
(60) Provisional application No. 60/838,587, filed on Aug. 17, 2006.

(51) Int. Cl. C12Q 1/68

(52) U.S. Cl. 435/6

(57) ABSTRACT

The present invention provides methods for identifying and evaluating suites of biochemical and/or gene entities useful as biomarkers for early prediction of disease and/or toxicity, disease staging, target identification/validation, and monitoring of drug efficacy/toxicity. The present invention further provides suites of small molecule entities as biomarkers for non-invasive and early prediction of hepatic injury.

### CROSS-REFERENCE TO RELATED APPLICATIONS

This application claims the benefit of U.S. Provisional Application No. 60/838,587, filed Aug. 17, 2006, the entirety of which is hereby incorporated by reference herein.

### STATEMENT REGARDING FEDERALLY SPONSORED RESEARCH

This invention was made with United States Government support under Cooperative Agreement No. 70NANB2H3009 awarded by the National Institute of Standards and Technology (NIST). The United States Government has certain rights in the invention.

### FIELD OF THE INVENTION

The present invention relates to methods for identifying biomarkers useful for early prediction of disease and/or toxicity, disease staging, target identification/validation, and monitoring of drug efficacy/toxicity. Biomarkers of the invention comprise one or more small molecule metabolites and/or differentially expressed genes, useful for early detection of hepatic injury.

### BACKGROUND OF THE INVENTION

Biological markers (biomarkers) are measured characteristics of an organism that correlate with normal or pathogenic biological processes, such as a particular disease state or toxic effect, or lack thereof. As a result, biomarkers are useful for the diagnosis and treatment of disease, and for predictive toxicology. Over the past 200 years, the quantitative chemical analysis of body fluids has steadily evolved to become a critical part of medical diagnosis and patient care (Porter, R. “The Greatest Benefit to Mankind,” WW Norton and Company, New York, 1997). Today, clinical laboratories routinely provide data on more than 200 biologically relevant analytes in blood and urine that can signal serious illness and monitor well-being. However, it has become increasingly clear that single markers (e.g. glucose) do not provide adequate information about complex diseases, and that composite biomarkers are much more likely to be of prognostic value.

There is currently renewed interest in the identification of biomarkers for staging clinical diseases, providing insights into mechanisms of drug action and predicting toxicity. Recent examples of the use of biomarkers for the prediction of disease and toxicity include U.S. patents: U.S. Pat. No. 6,540,691, “Breath test for the detection of various diseases;” U.S. Pat. No. 6,537,744, “Use of biomarkers in saliva to evaluate the toxicity of agents and the function of tissues in both biomedical and environmental applications;” U.S. Pat. No. 6,500,633, “Method of detecting carcinomas;” and U.S. Pat. No. 6,465,195, “Predictive diagnosis for Alzheimer's disease.” Hepatic toxicity is of particular importance, as it accounts for approximately 80% of toxicological failures in pre-clinical studies of potential pharmaceutical agents. In addition, liver toxicity is the major reason for failure of new chemical entities in clinical trials and the major reason existing drugs are pulled from the market.

The present invention provides methods for the identification and use of biomarkers for disease and/or toxicity, disease staging, target identification/validation, and monitoring of drug efficacy/toxicity. Biomarkers are provided for use as a non-invasive and early predictor of hepatic toxicity.

### SUMMARY OF THE INVENTION

The present invention provides methods for the identification and evaluation of small molecule and gene expression biomarkers of disease and/or toxicity and provides suites of small molecule entities as early predictive biomarkers of liver injury. Such biomarkers have important uses, including staging of disease and monitoring of drug safety and efficacy. The methods of the invention include the use of metabolomics and gene expression profiling to monitor a large number of low molecular weight biochemicals and genes, respectively. Such analysis reveals biomarkers and biomarker suites that discriminate among test groups. In one embodiment of the invention, the identified biomarker suites are linked to defined biochemical pathways and visualized using a ‘systems biochemistry’ view. In another embodiment of the invention, the biochemical entities identified according to the methods of the invention are provided for use as early biomarkers in serum and/or urine of liver damage and/or disease.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Principal component analysis (PCA) of rat liver biochemical profiling data collected for a study of acetaminophen-induced liver toxicity. Rats administered a single dose of acetaminophen (APAP) at 150, 1500 or 2000 mg/kg p.o. were sacrificed at 6 hr (squares), 18 hr (circles), 24 hr (triangles), or 48 hr (stars) post dosing (6 rats per group). The 150 mg/kg dose is equivalent to a low overdose level in humans (˜10 g) and 1500 mg/kg is a low toxic dose in rats. Livers of the rats were processed for biochemical profiling data and the third principal component (x-axis) is plotted against the second principal component (y-axis) for each of the 6, 18, 24 and 48 hr time points at 150 mg/kg (shapes with white fill), 1500 mg/kg (shapes with black fill), and 2000 mg/kg (shapes with striped fill) concentrations of acetaminophen. Each data point represents the average for six animals.

FIG. 2. A visualization of an identified subset of compounds whose relative responses follow trends observed for acetaminophen-induced changes in rat liver. The diagram displays that each of the identified compounds belongs to one of three interconnected metabolic pathways including purine metabolism, urea cycle and phenylalanine metabolism. In the diagram, a circle containing a KEGG compound identifier represents each of the compounds. The color red (designated as “R”) versus the color green (designated as “G”) represents whether the relative response of the compound increased or decreased in response to the acetaminophen-induced toxicity, respectively. Compounds that were not analyzed in the biochemical profiling study are designated as yellow (designated as “Y”) circles. Compounds whose relative responses were not statistically different from control are in blue (designated as “B”).

FIG. 3. A graph of response relative to control in liver (standard difference; y-axis) versus time from acetaminophen (2000 mg/kg) dosing (x-axis) for each of the 24 peaks listed in Table III.

FIG. 4. A graph of response relative to control in urine (standard difference; y-axis) versus time from acetaminophen (1500 mg/kg) dosing (x-axis) for each of the 24 peaks listed in Table III.

FIG. 5. A graph of response relative to control in serum (standard difference; y-axis) at 48 hours after acetaminophen (1500 mg/kg) dosing for each of the peaks listed in Table III (x-axis).

FIG. 6. A graph of response relative to control in liver tissue (standard difference; y-axis) versus time from acetaminophen (2000 mg/kg) dosing (x-axis) for each of the 7 peaks listed in Table IV.

FIG. 7. A graph of response relative to control in urine (standard difference; y-axis) versus time from acetaminophen (1500 mg/kg) dosing (x-axis) for each of the 7 peaks listed in Table IV.

FIG. 8. A visualization of an identified subset of compounds (Candidate Biomarker Suite II) whose relative responses follow trends observed for acetaminophen-induced changes in rat liver. In the diagram, a rectangle containing a KEGG compound identifier represents each of the compounds. The color red (designated as “R”) versus the color green (designated as “G”) represents whether the relative response of the compound increased or decreased in response to the acetaminophen-induced toxicity, respectively. Suite II contains 26 peaks, 21 of which were mapped to a KEGG identifier. 12 of these identified compounds map to a single subcluster, with 53 total compound nodes. The remainder of the identified compounds were mapped to disconnected singletons. The sub-network shown in FIG. 8 comprises parts of purine and pyrimidine metabolism, alanine and aspartate metabolism, and a branch leading to glycerolipid metabolism at the bottom of the figure.

FIG. 9. A visualization of an identified subset of compounds (Candidate Biomarker Suite II) whose relative responses follow trends observed for acetaminophen-induced changes in rat liver. In the diagram, a rectangle containing a KEGG compound identifier represents each of the compounds. The color red (designated as “R”) versus the color green (designated as “G”) represents whether the relative response of the compound increased or decreased in response to the acetaminophen-induced toxicity, respectively. Suite II contains 26 peaks, 21 of which were mapped to a KEGG identifier. 12 of these identified compounds map to a single subcluster, with 53 total compound nodes. Compounds whose relative responses were statistically different from control are highlighted in blue (designated as “B”). The remainder of the identified compounds were mapped to disconnected singletons. The sub-network shown in FIG. 8 comprises parts of purine and pyrimidine metabolism, alanine and aspartate metabolism.

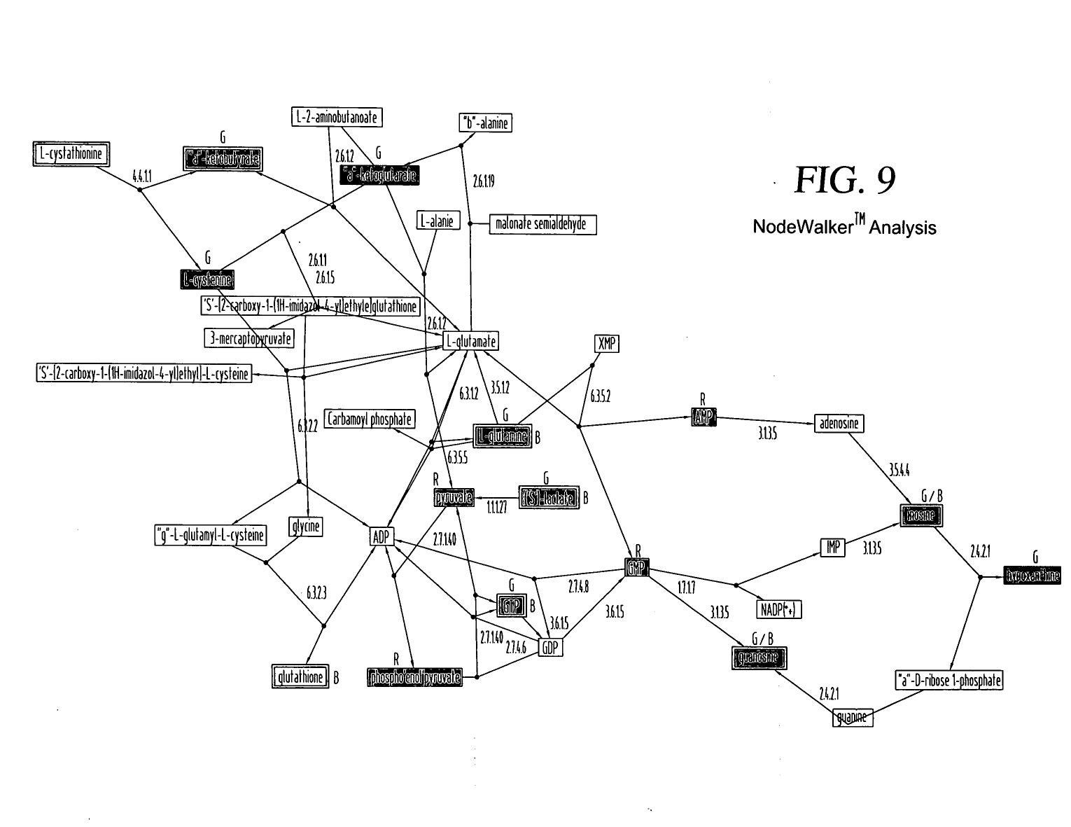

FIG. 10. A visualization of the metabolic network shown in FIG. 9, in which purine metabolism is highlighted in yellow (also designated using double lines). In the diagram, a rectangle containing a KEGG compound identifier represents each of the compounds. The color red (designated as “R”) versus the color green (designated as “G”) represents whether the relative response of the compound increased or decreased in response to the acetaminophen-induced toxicity, respectively. Compounds whose relative responses were statistically different from control are highlighted in blue (designated as “B”).

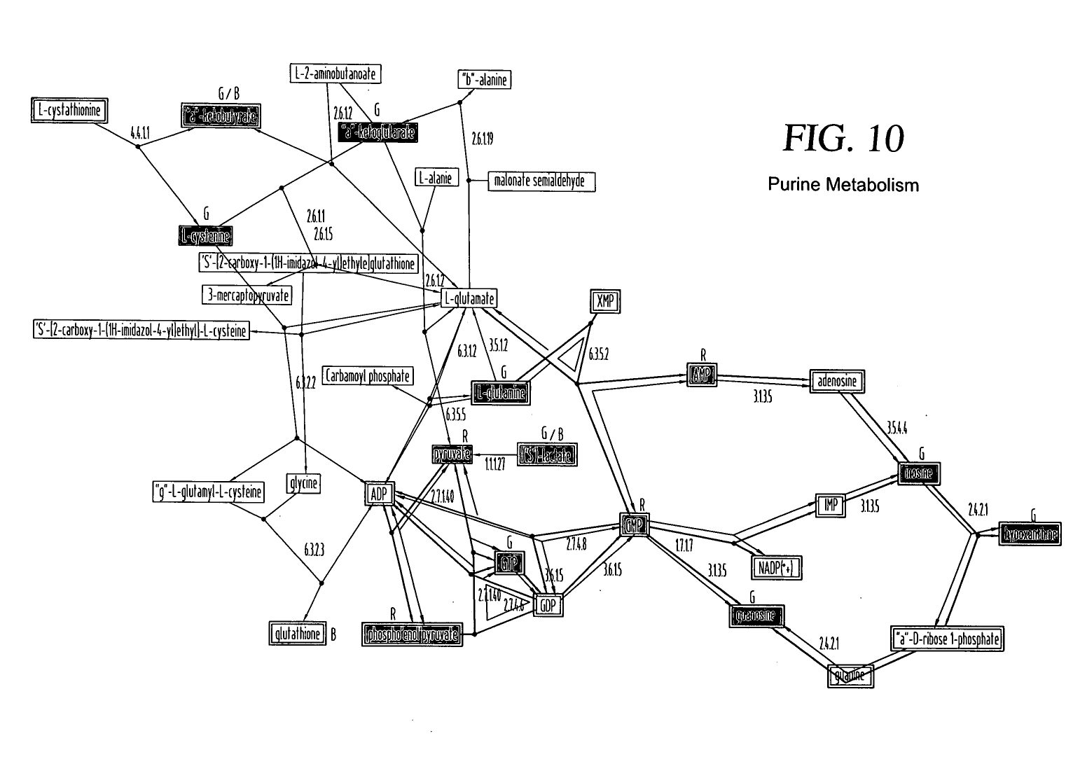

FIG. 11. A visualization of the metabolic network shown in FIG. 9, in which glutamate metabolism is highlighted in yellow (also designated using double lines). In the diagram, a rectangle containing a KEGG compound identifier represents each of the compounds. The color red (designated as “R”) versus the color green (designated as “G”) represents whether the relative response of the compound increased or decreased in response to the acetaminophen-induced toxicity, respectively. Compounds whose relative responses were statistically different from control are highlighted in blue (designated as “B”).

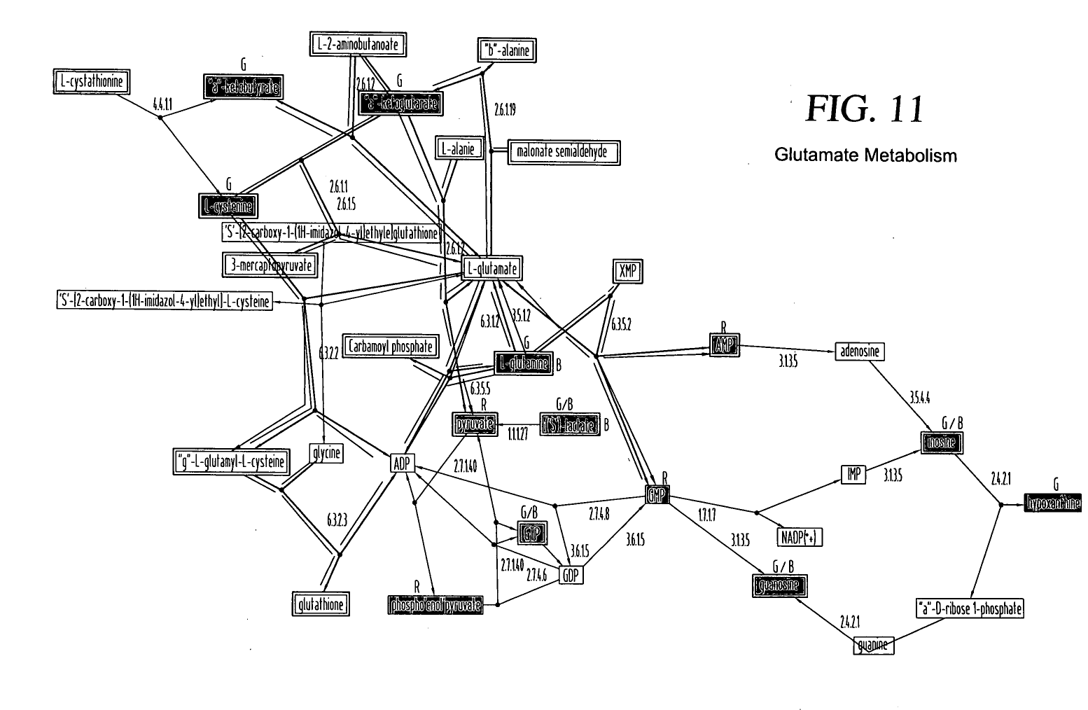

FIG. 12. Diagram displaying the gene expression profiling results of unsupervised clustering of genes that are present and variable across liver tissue samples from rats dosed with acetaminophen. Clusters A and B are outlined in white boxes and indicate that a different set of genes are overexpressed relative to control in an early versus a later response to dosing with 2000 mg/kg APAP.

FIG. 13. A graph showing the biological functions of the genes identified as being present in Cluster A from FIG. 12. The majority of genes in Cluster A are involved in metabolism of amino acids and nucleotides. The early transcriptional response to APAP-induced liver changes is consistent with changes in oxidative stress that were observed in the metabolite profile (see, in particular, FIG. 2 and FIGS. 8-11).

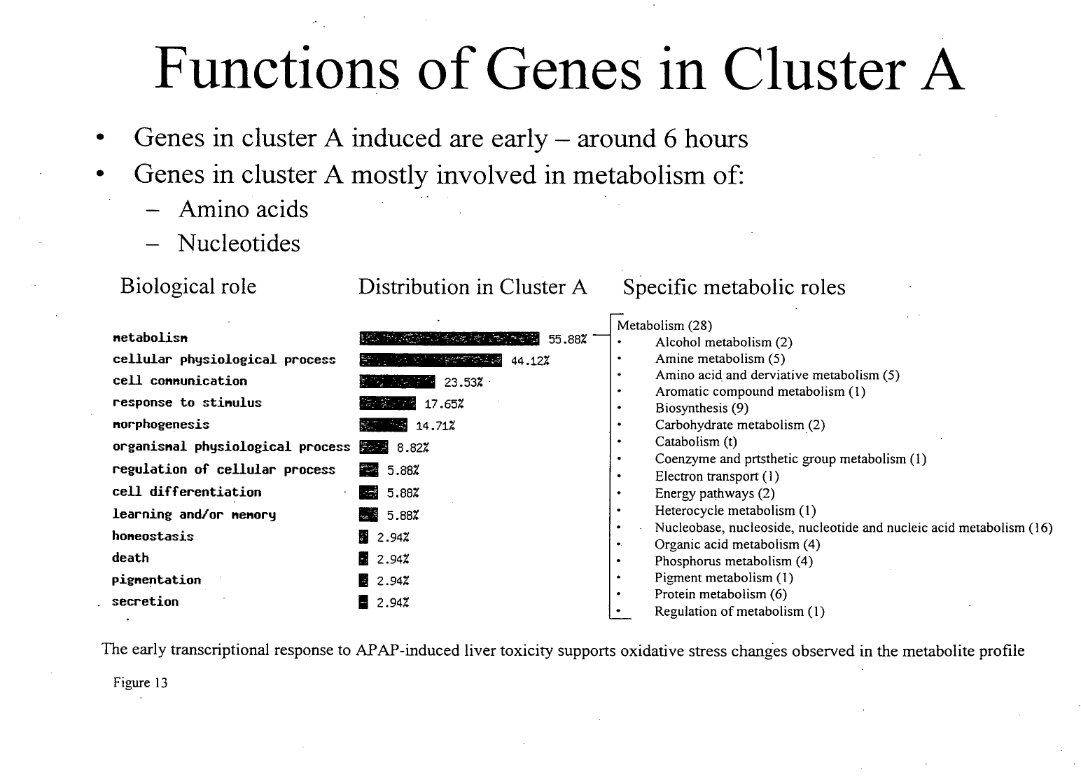

FIG. 14. A graph showing the biological functions of the genes identified as being present in Cluster B from FIG. 12. The majority of genes in Cluster B are involved in metabolism of amino acids and nucleotides and reactive oxygen species (ROS). The later transcriptional response to APAP-induced liver changes is consistent with changes in oxidative stress that were observed in the metabolite profile (see, in particular, FIG. 2 and FIGS. 8-11) and includes induction of ROS genes.

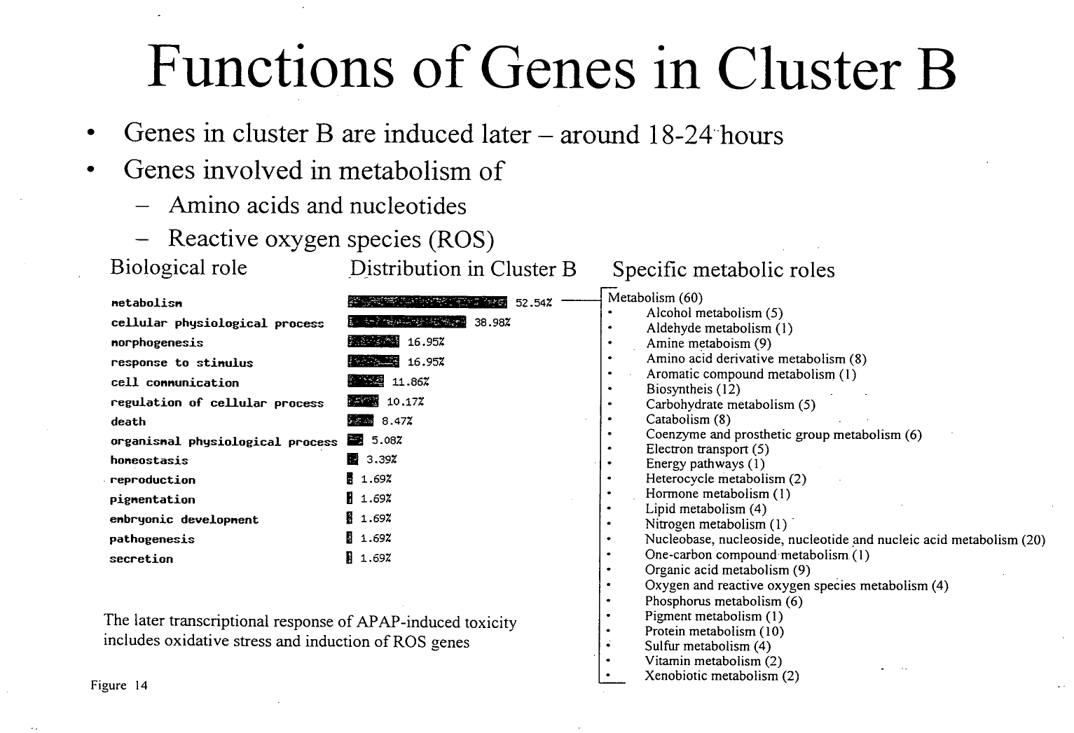

FIG. 15. Summary description of the effects on gene expression in liver of rats dosed with acetaminophen.

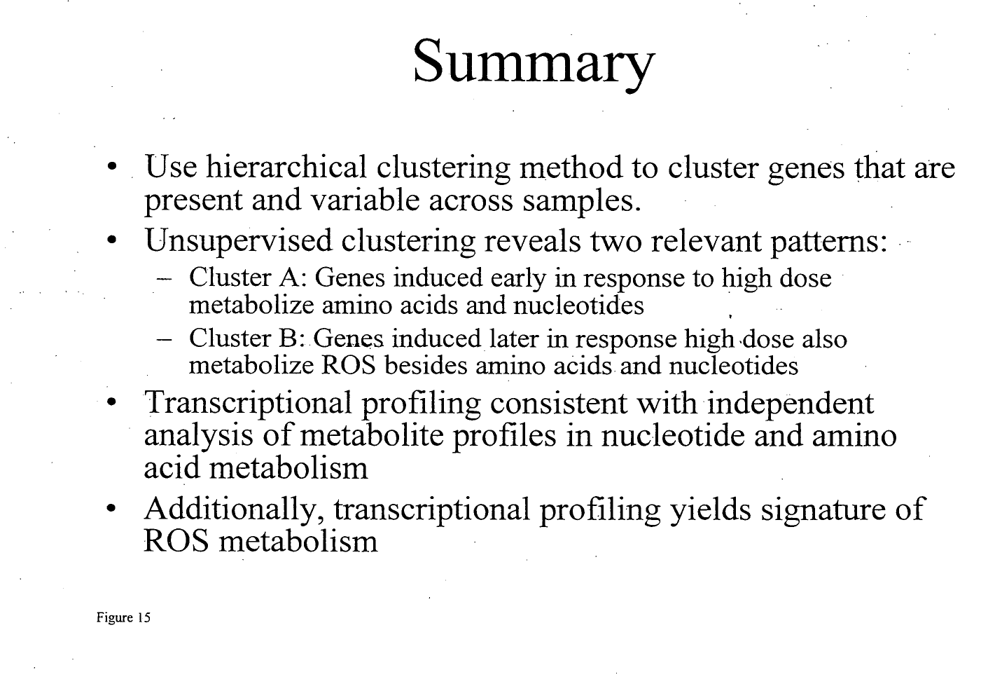

### DETAILED DESCRIPTION OF THE INVENTION

The present invention provides methods for the identification of small molecule and gene expression biomarkers of liver injury (disease and/or toxicity) and provides a suite of small molecule entities for use in early detection of such liver injury. Small molecule biomarkers of liver injury have a range of important uses, including staging of disease, monitoring of drug safety and efficacy, and target identification/validation. The methods of the invention include the use of metabolomics and gene expression profiling (GEP) to monitor a large number of low molecular weight biochemicals and genes simultaneously. In one embodiment, suites of the biochemicals are identified and objectively evaluated using statistical and biological metrics. Such analysis reveals biomarker suites that discriminate between test/experimental groups. Biochemicals are identified and objectively evaluated using statistical and biological metrics. In another embodiment of the invention, clusters of differentially expressed genes are identified using statistical and biological metrics. In another embodiment of the invention, the identified small molecules and/or differentially expressed genes are linked to defined biochemical pathways and visualized using a ‘systems biochemistry’ view.

Metabolomics, or biochemical profiling, provides a functional snapshot of system level responses and a window into less accessible genetic/cellular events. Resulting metabolomic data can be combined with other data streams (e.g. phenotypic analysis, such as tissue feature analysis, gene expression profiling, proteomics, etc.) to gain information beyond what can be resolved using a single platform. The strength of the metabolomics approach lies in the convergent nature of metabolism, involving a limited number of defined ‘players’ that can be globally profiled and linked to biological function at the level of cell and system. The biomarker suites of the present invention bridge experimental data to clinical outcomes; bridge gene, cell and/or tissue events to more accessible peripheral biomarkers; and build disease knowledge and support drug research and development (i.e. biomarker-enabled drug discovery).

The invention provides data analysis methods for identifying and evaluating a subset of a biological dataset as a biomarker of a biological phenomenon of interest. In one embodiment of the invention, metabolomic data are analyzed by one or more statistical means such as principal components analysis, discriminant analysis, cluster analysis and standard difference analysis to identify a subset of the data that distinguishes between experimental groups. In this manner, the subsets of biochemicals or genes or phenotypic features whose levels correlate with the progression of a disease or biological effect of interest are identified. The subsets of data points that best distinguish between experimental groups are identified as candidate biomarker suites. In a particular aspect of this embodiment, the methods of the invention are useful for identifying a subset of molecular entities as a candidate biomarker suite, whose relative presence in a target tissue is indicative of a biological phenomenon taking place in another less accessible tissue.

In one example, metabolomic data are analyzed using an algorithm to locate a subset of the data that exhibits a particular trend of interest across dose and time or a trend that correlates with surrogate- or clinical-endpoints of interest. In one embodiment, stepwise discriminant analysis (SDA) is performed on the located subset, to identify a second subset that best distinguishes between doses and times or between classes of a surrogate/clinical endpoint. In one example, the clinical endpoint is a pathologist's rating of a liver tissue slide. The subset of data points that best distinguishes between experimental groups is identified as a candidate biomarker suite. In another example, the data are analyzed using an algorithm to locate a subset of the data that exhibits a linear trend across dose and a linear or quadratic (but not higher-order) trend across time.

Given a large set of measurements on compounds, genes, proteins, etc., it is challenging to locate the subset of most interest in a particular experiment. Several statistical methods exist for analyzing such data that include: 1) Examining the measurements one at a time and choosing all of the measurements that are statistically significant. This method is too general in that it fails to distinguish between results that are relevant to the issue being studied and those that are not relevant to the particular issue. 2) Using discriminant analysis (or some other classification procedure) alone to choose the set of measurements that distinguish between experimental groups. This method is also too general because some experimental groups may, in fact, not be greatly affected by the experiment. Attempting to distinguish these groups from each other causes the method to include many measurements that are irrelevant to the problem. 3) Using prior knowledge of the treatment mechanism or disease mechanism to look for expected and inferred perturbations of individual molecules or entities. This method is too specific, as it only looks for what is expected and will not lead to new discoveries, except in the negative sense. Thus, existing methods generally provide too much information to the user, or not enough.

The data analysis methods of the present invention address each of the foregoing problems by using more information than the other methods, in the form of user input on what kinds of trends are of interest for a particular dataset. Once a trend has been designated for a dataset, the methods of the invention enable location of a subset of the data points that corresponds to particular molecular entities that match the designated trend. In this manner, one or more molecules are discovered to be associated with a biological phenomenon even though the trend for the particular molecule(s) may not have been evident in the dataset as a whole. The methods of the invention allow for identification of molecular entities that are involved in a biological phenomenon of interest, entities that may have otherwise gone undiscovered in a complex dataset.

In one embodiment, the present invention provides data analysis methods for the rapid location of subsets of complex biological datasets that are of most interest for further analysis. The biological datasets of the invention include, but are not limited to; data obtained or collected using well known techniques such as biochemical profiling, gene expression profiling, and protein expression profiling, as well as other techniques such as tissue feature analysis. Tissue feature analysis refers to quantitative tissue image analysis of structural features in tissue elements using digital microscopy to generate data that objectively describes tissue phenotype, with the potential for detection of subtle changes that are undetectable to the human eye as described, for example, in Kriete et al., 4 Genome Biology R32.1-0.9 (2003). The datasets for analysis using the methods of the invention comprise a baseline and one or more experimental groups. The methods of the invention allow a user to quickly pinpoint the subset of data of most interest without a concomitant loss of a large percentage of relevant information, as is typical with standard methods. In related embodiments described in more complete detail below, the invention provides data analysis methods for investigating the molecular mode of action of a biological phenomenon of interest and for identifying data points that best distinguish between experimental groups for use as biomarkers. In one aspect the invention is particularly useful for the identification and use of small molecule biomarkers of disease and/or toxicity, disease staging, target identification/validation, and monitoring of drug efficacy/toxicity.

In one embodiment of the present invention, a subset of data points is identified that correspond to a trend associated with a biological phenomenon of interest when the trend is evident through visual inspection of the dataset as a whole. Visual inspection of the dataset as a whole includes inspection of the dataset prior to or subsequent to the data being reduced or otherwise manipulated, such as for example, through use of a two-dimensional plot of a first principal component vs. a second principal component, or other representation. In another aspect of the embodiment, a subset of data points is identified that corresponds to a trend associated with a biological phenomenon of interest when the trend is not evident by visual inspection of the dataset as a whole, even after data reduction or other manipulation. In this case, the trend is designated because it is a known or predicted trend for the biological phenomenon of interest even though the trend is not evident in the dataset as a whole. A related embodiment is when the trend of interest is evident in another dataset, for example a dataset from another tissue, but the trend is not evident in the dataset for the tissue being targeted.

Thus, in one embodiment, the present invention provides data analysis methods for locating subsets of large, multivariable biological datasets that are of most interest for further analysis. The methods of the invention comprise the steps of: a) obtaining a set of data points for a biological phenomenon of interest wherein the set of data points comprises a baseline and one or more experimental groups; b) designating a trend in the set of data that is associated with the biological phenomenon of interest; c) developing a mathematical model of the trend; and d) testing each data point in the set for adherence to the mathematical model, wherein the data points adhering to the model are the subset of most interest for further analysis. In the methods of the invention, the designated trend may be observable in a display of the dataset as a whole or may instead be a known trend or a trend that is predicted to be associated with the biological phenomenon of interest even if it is not evident in the dataset as a whole. In preferred embodiments of the invention, the sets of data points comprise biochemical profiling data, gene expression profiling data, protein expression profiling data, and tissue feature data.

In another embodiment, the invention provides data analysis methods for investigating the molecular mode of action of a biological phenomenon of interest. The methods comprise the steps of: a) obtaining biochemical profiling data, gene expression profiling data, protein expression profiling data, or tissue feature data for a biological phenomenon of interest, wherein the data comprises a baseline and one or more experimental groups; b) designating a trend in the data that is associated with the biological phenomenon; c) developing a mathematical model of the trend; d) testing each data point in the set for adherence to the mathematical model; and e) identifying one or more metabolic pathways to which the data points that adhere to the model belong, wherein the mode of action of the phenomenon of interest affects the identified metabolic pathways. In the methods of the invention, the designated trend may be observable in a display of the dataset as a whole or may instead be a known trend or a trend that is predicted to be associated with the biological phenomenon of interest, even if it is not evident in the dataset as a whole.

Another embodiment of the invention is directed to identification of subsets of data points that best distinguish between experimental groups as putative biomarkers. The invention provides data analysis methods for locating a set of biological data points as a biomarker of a biological phenomenon of interest. The biomarkers of the invention have a range of uses including biomarkers of disease and/or toxicity, disease staging, target identification/validation, and monitoring of drug efficacy/toxicity. In a particular aspect of this embodiment, the methods of the invention are useful for identifying a subset of data points corresponding to molecular entities as a putative biomarker, wherein the relative presence of the subset of data points in a target tissue is potentially indicative of a biological phenomenon taking place in another less accessible tissue.

Thus, the methods of the invention are directed to identification of subsets of data points that best distinguish between experimental groups as putative biomarkers and the methods comprise the steps of: a) obtaining a dataset for a biological phenomenon of interest in one or more tissues that include a target tissue, wherein the dataset comprises a baseline and one or more experimental groups; b) designating a trend in the dataset that is associated with the biological phenomenon of interest for one of the tissues; c) developing a mathematical model of the trend; d) testing each data point of the one tissue dataset for adherence to the mathematical model; e) processing the data points that adhere to the mathematical model using stepwise discriminant analysis (SDA) to identify a small group of the adhering data points that best distinguishes between the experimental groups of the one dataset, wherein the small group i) consists of the data points rated most highly by the SDA, ii) each of the data points in the small group varies significantly from the baseline in the one dataset, and iii) if the small group is not derived from the target tissue, each of the data points in the small group is detectable in the target tissue and varies significantly from the baseline in the target tissue dataset; and, optionally, f) performing parametric discriminant analysis on the small group to obtain a discrimination score that measures how well the small group distinguishes between the experimental groups of the one dataset, wherein the higher the discrimination score the better the indication of the small group as a putative biomarker in the target tissue for the biological phenomenon of interest. In preferred embodiments of the invention, the datasets comprise biochemical profiling data, gene expression profiling data, protein expression profiling data, and tissue feature data.

In a particular instance of the foregoing embodiment the one tissue and the target tissue are the same, such that the dataset for which the biological phenomenon of interest is obtained is for the target tissue. The target tissues of the invention are preferably those tissues that are most readily attainable in humans and animals, for example, tissues such as serum and blood, although the methods of the invention do not restrict any tissue from serving as a target tissue. Similar to the methods for locating subsets of most interest for further analysis and for investigating modes of action, the methods of the invention for discovering biomarkers also encompass designating a trend in the dataset for the target tissue that is either observable in a display of the dataset as a whole or instead is a known trend or a trend that is predicted to be associated with the biological phenomenon of interest. Optionally, parametric discriminant analysis is performed on the small group to obtain a discrimination score that measures how well the small group distinguishes between the experimental groups of the dataset. The higher the discrimination score the better the indication for the small group as a putative biomarker in the target tissue for the biological phenomenon of interest.

In another instance of the foregoing embodiment the trend of interest is designated in a tissue other than the target tissue, and the discriminating small group is indicated as a putative biomarker in the target tissue. The one tissue and the target tissue are not the same in this embodiment of the invention. Because the discriminating small group is identified in a tissue other than the target tissue, each of the data points in the small group must meet the criterion (iii) noted above of being detectable in and varying significantly from baseline in the target tissue in addition to the criteria of being rated most highly by the SDA and varying significantly from the baseline in the originating non-target dataset. Similar to that described for the previous embodiments, the designated trend in the non-target dataset is either observable in a display of the dataset as a whole or instead is a known trend or a trend that is predicted to be associated with the biological phenomenon of interest. Optionally, parametric discriminant analysis is performed on the small group to obtain a discrimination score that measures how well the small group distinguishes between the experimental groups of the target dataset. The higher the discrimination score the better the indication for the small group as a putative biomarker in the target tissue for the biological phenomenon of interest. Using the methods of the invention in this manner allows for identification of putative biomarkers that are measurable in readily accessible target tissues, biomarkers that might not otherwise be discoverable through sole analysis of data derived from the target tissue.

In another embodiment of the invention, a candidate biomarker suite is provided for early detection and monitoring of liver damage and or disease in serum and/or urine. Early identification of liver injury is important, as the liver performs a central role in diverse range of metabolic processes including intermediate metabolism of amino acids, synthesis and degradation of proteins, regulation of cholesterol and lipid metabolism, and metabolism and degradation of drugs and hormones. Furthermore, liver toxicity has high medical/pharmaceutical relevance, as drug-induced hepatotoxicity is a key regulatory issue and liver is directly involved in variety of disease states.

The biomarkers of the invention were identified through analysis of biochemical profiling data from tissue samples of rats in an acetaminophen dose-response and time-course study. Acetaminophen is a common over-the counter pain reliever (e.g. TYLENOL) and the leading cause of acute liver failure. Samples analyzed included urine, serum, and liver and resulted in the discovery of biomarkers for use in non-invasive and early prediction of liver toxicity. The following small molecules are provided for use as non-invasive and early predictive biomarkers of liver injury by analysis of a subject's urine or serum for the relative response of the small molecules.

Twenty six chemical entities for which a combined relative response in liver, serum and urine is indicative of liver damage/toxicity are listed in Table III (Candidate Biomarker Suite II). The relative response of the foregoing biochemical metabolites, measured according to the methods of the invention, is indicative of liver damage and, thus, useful as a non-invasive biomarker for the early detection/diagnosis of liver toxicity and/or disease. Measurement of relative response in serum or urine for any combination of one or more of the response peaks listed in Table III may also be useful as an early biomarker of liver damage and/or disease. The combinations of response peaks having the highest explanatory power of the observed trend and statistically significant changes among dose/time groups in the target tissue are the preferred combinations for biomarker use. In the methods of the invention, response relative to control is measured, for example, by use of gas or liquid chromatography followed by mass spectrometry. However, any technique for quantifying metabolite response relative to control, known to those of ordinary skill in the art, is applicable to the methods of the present invention.

In another embodiment of the invention, a biomarker suite in urine is provided for early detection and monitoring of liver damage and or disease. Six chemical entities for which a combined relative response in urine is indicative of liver damage/toxicity are listed in Table IV with the exception of Peak13, and are as follows: aspartic acid, Peak12, L-glutamine, Peak20, succinic acid, and Peak26. The relative response in urine of the foregoing biochemical metabolites, measured according to the methods of the invention, are indicative of liver damage and, thus, useful as a non-invasive biomarker for the early detection/diagnosis of liver toxicity and/or disease. In one aspect of the invention, liver damage/disease is indicated by measurement in urine of a standard difference in response relative to control of greater than or equal to 2 for each of the above listed metabolites. The response relative to control in urine for metabolites, L-glutamine and Peak20, is a positive response that is greater than or equal to 2, whereas the relative response for aspartic acid, Peak12, succinic acid, and Peak26 is a negative response of greater than or equal to 2 relative to control. Response relative to control is measured, for example, by use of gas or liquid chromatography followed by mass spectrometry. However, any technique for quantifying metabolite response relative to control, known to those of ordinary skill in the art, is applicable to the methods of the present invention.

In another embodiment of the invention, biochemical entities whose levels or responses correlate with the progression of a disease or biological effect of interest are mapped to biochemical pathways. Pathway linkage moves beyond simple classification to mechanism, and addresses the questions of why and how a particular effect occurs. NODEWALKER is a query and analysis method the invention, used for identifying patterns in experimental data. NODEWALKER provides meaning or context for experimental data, including metabolic, gene expression and proteomic data, by applying the data to a network representation of biological processes. In doing so, NODEWALKER moves beyond the traditional pathway view of biology to a network view, and uses the network as a data integration tool to seamlessly merge disparate data streams. The NODEWALKER tool is designed to answer key questions a researcher might have about an empirical data set, such as what part of the metabolic network has been perturbed by a particular treatment or state change, or do compounds, genes or proteins that can be related by clustering or correlation analysis of the empirical data also lie close to one another in the network.

As biology has traditionally been carried out, a researcher measures a relatively small set of variables relating to a system, and due to the small size of the data set, is able to reach meaningful conclusions by manual examination of the data. In the systems biology approach where, for example, a substantial fraction of the transcriptome and the metabolome are measured across as dose and time series, the resulting dataset is far to complex to be mined successfully with existing tools. NODEWALKER acts as a data simplification tool by applying the experimental data to a network representation of biology, with the aim of identifying relationships that would not otherwise have been evident in the raw data. The purpose of NODEWALKER is to identify perturbed regions of the network, and as such NODEWALKER is both an explanatory and a hypothesis-generating tool.

In the methods of the invention, NODEWALKER functions in one of two modes designated as “naïve mode” and precomputed cluster mode” to generate a graph of interconnected metabolic pathways related to one or more perturbed biochemicals, genes or proteins.

Naïve Mode:

1) Start with a list of perturbed nodes (“node” refers to any one of a metabolite, gene or protein).

2) Place the first nodes in the center of a new graph.

3) Find all nodes in the database (i.e. KEGG, BRENDA, etc., or any database of metabolic pathways) that are one reaction away from the starting compound. Add these to the graph.

4) Continue this process for each of the new nodes on the graph. Repeat until a predetermined search depth (number of reactions removed) is hit.

5) Each time a nodes in the input file is found, remove it from the “to do” list.

### Precomputed Cluster Mode:

If a relationship has been identified among a set of nodes in the empirical data (for example through clustering or correlation analysis), these sets can be directly used by NODEWALKER as follows:

1) Start with a list of related nodes.

2) Load a graph representation of the network into memory.

3) For each node in the set, determine if there exists a path connecting it to each other node in the set. If a path shorter than some cutoff length is found, return this path.

4) Add the resulting paths to a new graph.

At the end of the procedure, nodes from the input set will reside in one or more disconnected subgraphs, with the exception of “singleton nodes” for which no path shorter than the cutoff could be found that would connect them with the rest of the set. At the end of either of these procedures, the resulting subgraphs will have annotation information applied to them to determine which canonical pathways, i.e., those familiar to biologists through textbook representations, are spanned by the new subgraphs. Finally, a perturbation score is applied to each identified subgraph. The score is used either to rank identified subgraphs in order of importance, or to track changes in perturbation level for a region of the network across a dose and time series.

One factor that increases the complexity of the metabolic network is that the network is dominated by “supernodes,” compounds like water, ammonia, or ATP, that are involved in hundreds or thousands of reactions. The supernodes distort the network structure, and will distort actual pattern discovery in the network. For example, ATP is a substrate in 414 reactions in KEGG and a product in 17 reactions. ADP is a substrate in 18 reactions in KEGG and a product in 298 reactions. One solution to the distortion caused by supernodes is to filter compounds by connectivity. Compounds are sorted by number of reactions so that a set of compounds having a very large number of connections is found that can be unambiguously filtered out, e.g. water, ammonia, carbon dioxide, chloride anion, etc. These compounds are unambiguous because there is never an instance in which they represent flux through a biosynthetic pathway. The problem is that as one proceeds further down the list of high connectivity compounds, “mixed role” compounds begin to appear. The solution is use of a database that includes precise reaction role information for each compound in each reaction. For example, information describing the role of glutamine as a simple amino donor in some reactions, but a legitimate substrate or product in other reactions, must be supplied by the underlying database.

NODEWALKER approaches the analysis of metabolic networks in the following way. Patterns (like control points) in metabolic networks rely on distance between nodes. Distance through trivial nodes is usually shorter than actual biosynthetic distance. Some compounds (like water) are always trivial. Others (like glutamine) are sometimes trivial and sometimes key players. The network is only meaningful if connections between nodes represent actual biological events. NODEWALKER can be used to identify perturbed portions of the metabolic network, to identify control points in particular pathways, to identify therapeutic targets, and to identify biomarkers and/or suites of biomarkers.

In another embodiment of the invention, the performance of a given candidate biomarker suite is objectively evaluated using the following statistical and biological metrics. The first metric is referred to as “Accuracy” which is a generally known term defined as the percentage of correct classification achieved on the original data set, wherein classification is any user determined endpoint such as, for example, clinical endpoint, surrogate endpoint, or experiment/treatment. The Accuracy method generally over-estimates the accuracy of the classification approach applied to a new data point from the same population, since the accuracy is measured on the same dataset from which the discriminant rule was derived.

The second metric is known as “Cross-validation Accuracy” and is a generally known method that yields a more objective assessment of Accuracy. Cross-validation Accuracy is defined as the percentage of correct classifications using different training and testing data sets. The approach generally known as “leave-on-out” is used in the invention to calculate the Cross-validation Accuracy. In the foregoing approach, the training and testing data is formed by dividing the dataset into training and testing sets by taking one instance as a test sample and the remaining (N−1) samples as training examples. The training data is used to induce the discrimination function and the test instance is used to calculate the classification accuracy. The method provides an unbiased estimate of the accuracy of the classification rule applied to a new data point from the same population.

The third metric is referred to as “Confusion Score” and the Score for a given biomarker suite is calculated as the average distance between an observation's true class and the class into which it was classified (see “Accuracy” above). The average is calculated over all observations in the original dataset. As such, it provides additional information to the accuracy score, because the Confusion Score will increase as the classification moves further away from the true class. The distance metric used for calculation of the Confusion Score is Manhattan (a.k.a. “city-block”) distance. That is, a distance of 1 separates any two adjacent classes.

### Formally,

let C be the classification function
let Sᵢⱼ be the jth subject in the ith treatment group.

Let n be the number of subjects
Let C(Sᵢⱼ)=cᵢⱼ
Let ∥x,y∥ denote the Manhattan distance between x and y.

Let dij=∥i,cᵢⱼ∥

Then the Confusion Score=1/nΣΣdij

The fourth metric is referred to as “TRENDFINDER Score” and the score for a given biomarker suite is calculated from the p-values assigned to the peaks in the algorithm of the trend of interest. The score is calculated as follows:
The algorithm of the trend of interest contains a mathematical model in one or more features (such as dose, time, blood pressure, pathology score, radiology score, etc.)
These features can either be desired features of the model, or undesired features (i.e. trends that would lead the user to remove data from further consideration). For example, it might be the case that a negative linear trend in blood pressure is a desired feature, whereas a positive linear trend is an undesired feature of the model.
Each peak is assigned a p-value for each term in the model: pᵢⱼ=p-value for ith term in the model and the jth compound.
dᵢ=1 if the ith term is desired, and 0 if it is undesired.
zᵢ=p-value cutoff for significant undesired term i. (E.g. zᵢ=0.01).
wᵢⱼ=1 if the pᵢⱼ≦zᵢ, and 0 otherwise.
S₁ⱼ is calculated as the sum of the p-values for desired features: S₁ⱼ=Σdᵢpᵢⱼ
S₂ⱼ is calculated as the sum of the p-values for significant undesired features: S₂ⱼ=Σ(1−dᵢ)pᵢⱼwᵢⱼ
Sⱼ=S₁ⱼ−S₂ⱼ
S=average Sⱼ over all compounds
The trendFinder score=100(1−S).
   This score will be larger for compound sets that more closely match the trend.

The last metric used in evaluating candidate biomarker suites is degree of biological relevance. Biochemical entities of the candidate biomarker suite are mapped to biochemical pathways through computational inference using the NODEWALKER technology described herein, supra. The NODEWALKER results indicate where the metabolic network has been perturbed and whether the biochemical entities of the biomarker suite lie close to one another in the network. A high degree of biological relevance is indicated when the perturbations are clustered together in regions of metabolism that are predicted by the disease/treatment state.

The methods of the invention comprise generation of information management methodologies (data integration). In one embodiment of the invention, a number of very different data sources are successfully integrated, including information on metabolism, enzymology, genome annotation, as well as empirical data including gene expression, biochemical profiling and phenotype analysis such as histomorphometry. Data types and data sources are identified for integration in one case by finding specific sources for each data type. An integration approach is designed for each data type. As a general rule, the preference is for all data to reside within a single schema, rather than to use data warehouse or federated database approaches, but this decision is made with the specifics of each data source in mind.

In the methods of the invention, gene function data are integrated and represented. First, empirical and reference data are integrated to assist in understanding experimental results. This is in a logical structure that enables the entire corpus to be interrogated through a common interface, or by a single analytical tool. A robust relational database structure is used to manage large-scale biological data and ontologies to represent the complex structural and functional descriptions of living systems.

In addition to large-scale biomolecular assays, histomorphometric (HMP) data represents a novel and highly complex form of biological information that is integrated into a common, logical system for the creation of coherent data sets. Such tissue images are a rich informational resource that can be utilized to assess regional variations in tissue structure and function. All information from HMP will be integrated into the database structure. In the methods of the invention, data integration is performed a priori, through database development with integration, rather than a posteriori, attempting to forge sensible links between separate, mature databases. Thus, a single database structure represents all of data streams, linked by study or sample identifier. Reference databases are integrated on a case-by-case basis. Database schemas are designed to represent this information, which will be integrated with the empirical data through logical mappings (for example compound or gene hubs).

In the methods of the invention, an ontology is a specification of a conceptualization. That is, it is a formal specification for representing all relevant information about a specific domain of interest. In biology, ontologies have become invaluable for expressing complex concepts and for making such concepts amenable to computational manipulation. At a simple level, an ontology provides a controlled vocabulary to define a domain of knowledge, such as gene function or compound mode of action. At a more complex level, an ontology is a complete semantic framework for organizing knowledge about a complex domain. The methods of the invention comprise use of different types of ontologies.

In the methods of the invention, preparation of complex biological information for mining empirical relationships involves object “flattening.” This refers to the transformation of complex objects to vectors or attribute-value tuples. Object data are converted (flattened) into a nominal or ordinal representation to enable subsequent data processing and consolidation. Information linkages back to the raw data are established to enable increasingly complex levels of data analysis.

In one embodiment of the invention, Laboratory Information Management Systems (LIMS) are used to facilitate data capture and storage, allowing the automation of many routine data management and processing tasks. Tools for integrating multiple platform-specific LIMS into a common framework are used for the data integration system. The tools manage data coming from each type of LIMS and enable transfer of data among the LIMS. The tools include validation of data transfer (sending and receiving) by following common LIMS rules for sample handling. Automatic control mechanisms are provided to alert users about errors and to protect data integrity. In one embodiment of the invention, Medical Subject Headings (from the National Library of Medicine), also known as MeSH terms for organisms and species are implemented at the point of sample accessioning. In another embodiment, archiving procedures for phenotypic data are formulated for parts of a particular phenotypic platform. In the case of histomorphometric (HMP) data, image montage and/or thumbnail images are transferred to software, and mechanisms for the association of images with HMP numerical and text data are implemented. Automated tools support the thin client transfer of images and HMP.

In one embodiment of the invention, data from a study on the toxic effects of acetaminophen are integrated to elucidate mechanism of action. The data from two different molecular profiling platforms are generated including gene expression profiling (GEP), and biochemical profiling (BCP). Prerequisites for the data include that the data are generated from biological samples, that a baseline is established for the data, and that the variability of the data is understood. The data are analyzed by any one or more of a variety of statistical methods to identify the molecules and/or genes involved in the mechanism of acetaminophen-induced damage. In one embodiment of the invention, pathways involved in APAP-induced liver toxicity are curated. The BCP and GEP data are objectively evaluated as to extent of support for existing knowledge. In another embodiment of the invention, the TRENDFINDER tool is used to identify or evaluate the BCP and GEP biomarker sets that follow user-defined trends. The separate BCP and GEP biomarker suites are then combined, and the tool re-applied to extract a combined BCP/GEP biomarker suite.

In one embodiment of the invention, samples are analyzed from a study on APAP-induced liver toxicity in rats. Rats are treated with a single oral dose of APAP at 0, 150, 450 and 2000 mg/kg. Animals are sacrificed at 0, 6, 18, 24 and 48 hours, and liver samples are obtained for the analysis of biochemical profiles, gene expression, histopathology and histomorphometric profiling. Serum samples are also obtained at the above times for routine clinical chemistry and biochemical profiling. Urine is collected from the rats for biochemical profiling over the following time periods: 24 hours pre-dose, 0-6 hours, 6-24 hours and 24-48 hours. The foregoing is only one example of a study useful with the methods of the present invention, and the invention is not limited to such a study. One of ordinary skill in the art would understand that there are many studies useful with the methods on the invention.

In one embodiment of the invention, results from the foregoing samples (biochemical and transcriptional descriptors) are analyzed. Baseline levels are analyzed across all doses and times to determine the reproducibility of the control and treated samples using statistical methods. An analysis set is created by combining the BCP and GEP data, missing data are accounted for, and measurements are normalized. Data for each biological sample are loaded into a central database where it is accessible in an integrated fashion. The quality of the data is verified and it is further processed to ensure that the biochemicals and the transcripts measured for each sample are accurately comparable across the samples in the whole study. In one embodiment, significantly perturbed molecules are analyzed through parametric and non-parametric statistical methods. The analysis results in thousands of quantitative biomolecular descriptors (representing the levels of metabolites and of transcripts) for the 90 biological samples. The data set is analyzed with a broad array of analytical techniques.

In another embodiment of the invention, a coherent biological view of the mechanisms underlying the specific aspects of APAP-induced liver injury is elucidated, as well as the general processes of liver toxicity. Using a very high-dimensional data set is extremely challenging. A multi-strategy problem-solving approach is applied in light of the complexity of the causal mechanisms, the relatively small number of biological samples and the extremely high dimensionality of the available descriptors. Computational methods are used that are based on pattern classification (using multivariate statistics and machine learning) and automated biological reasoning to address this problem. Sets of descriptors (biomarkers) are discovered with higher-order correlations that accurately classify the liver samples in a biologically meaningful fashion, and the descriptor sets are evaluated in light of their biological interpretation. Evaluation comprises the use of metrics that indicate degree of statistical accuracy and significance for classification algorithms, and relevance scores for the biological validity of inferred mechanisms.

In another embodiment of the invention, multivariate statistical analysis and machine learning methods are used to classify clinical endpoints (or other biologically relevant phenotypes) using metabolite and transcript descriptors. A hybrid machine learning approach is used for descriptor subset selection and for the induction of complex decision boundaries for classifying the different clinical endpoints.

### EXPERIMENTAL

### Example 1

### Acetaminophen-Induced Liver Toxicity Study Design and Data Acquisition

An acetaminophen-induced liver toxicity study was performed as follows. Rats were administered a single dose of acetaminophen (APAP) at 0, 50, 150, 1500 or 2000 mg/kg p.o. (6 rats per group). The 150 mg/kg dose is equivalent to a low overdose level in humans (˜10 g) and 1500 mg/kg is a low toxic dose in rats. The rats were sacrificed at 6, 18, 24, and 48 hr post dosing. Livers of the rats were processed for biochemical profiling, histopathology and gene expression analysis. For the 0, 50 and 1500 mg/kg dose groups, rat urine was collected at −24-0, 0-6, 6-24, and 24-48 hr relative to dosing. Similarly, rat serum was collected at 48 hr for the 0, 50 and 1500 mg/kg groups.

### Sample Preparation

Rat tissue was prepared for LC-MS analysis as follows. Rat liver tissue samples were frozen upon collection. A slice of each frozen sample was placed into a mortar, covered with liquid nitrogen, and ground with a pestle. 100 mg of ground sample was placed in a cryovial, extraction fluid and beads were added, and the sample was further ground and then centrifuged. The supernatant was transferred to a clean cryovial, centrifuged again, and then transferred to a well of a 96-well plate.

Rat biofluids were prepared for LC-MS analysis as follows. Rat urine and serum samples were vortexed. An aliquot of 500 μL was transferred to a clean cryovial and centrifuged. The supernatant was transferred to a clean cryovial and centrifuged again. The supernatant was transferred to another clean cryovial and diluted 4:1 with extraction solvent. An aliquot of 100 μL was transferred to a well of a 96-well plate.

### LC-MS Analysis

LC-MS analysis of the rat tissue and biofluid samples was performed on an Applied Biosystems Mariner liquid chromatograph coupled with a time of flight mass spectrometer (LC-TOF). The mass resolution for the mass spectrometer employed in this experiment was 0.200 amu. One LC was employed with a splitter that allowed for the eluent to be delivered to two TOF MS instruments, one operating in positive ionization mode, the other in negative ionization mode. Compounds detected by LC-MS with an electrospray ion source were cataloged based on retention times and mass-to-charge ratio (m/z) of the ions characteristic of each peak. Mass spectrometric data were collected from 80-900 amu. Raw LC-MS data were processed using the commercially available software TARGETDB (Thru-Put Systems, Inc., Orlando, Fla.).

### RNA Isolation

Upon necropsy, liver tissue from left lateral lobe is cubed (0.5 cm or smaller) and stored in RNALATER (Ambion, Austin, Tex.) overnight at 4+/−3° C. then transferred to −20+/−10° C. until RNA isolation (within 60 days). RNA is isolated from approximately 130-150 mg tissue using RNEASY midi spin columns (Qiagen, Valencia, Calif.) according to the manufacturer's protocol. The RNA is concentrated using Millipore Microcon centrifugal filter devices (Billerica, Mass.).

### RNA Labeling/Microarray Hybridization

One μg total RNA from either an individual rat or a pooled sample is amplified and labeled with a fluorophore (either Cy3 or Cy5) using Agilent Technologies' Low RNA Input Linear Amplification Labeling (Palo Alto, Calif.) following the manufacturer's protocol. The resulting fluorescently labeled cRNA is tested on a Nanodrop ND-100 spectrophotometer (Rockland, Del.) and an Agilent Bioanalyzer (Palo Alto, Calif.) to ensure proper quantity and quality. Equal amounts (750 ng) of Cy3-labeled cRNA from an individual rat and Cy5-labeled cRNA from the corresponding pooled control are hybridized to an Agilent Rat Oligo Array (Palo Alto, Calif.). In a second hybridization, the fluorophores used to label each sample are reversed. Therefore, two hybridizations are performed for each individual rat examined in this study.

### Example 2

### Data Analysis Methods

### Locating a Subset of Most Interest that Corresponds to an Observed Trend

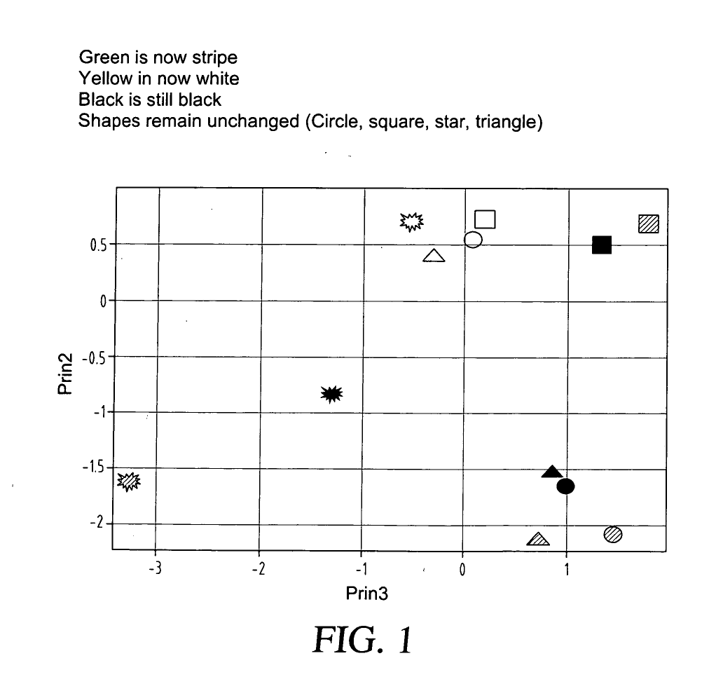

A targeted list of relative responses for peaks and peaks annotated as known compounds was produced from the LC/MS data for the rat liver samples described in Example 1. The resulting biochemical profiling data was subjected to Principal Component Analysis (PCA), showing trends over both dose and time (see FIG. 1). The third principal component (x-axis) is plotted against the second principal component (y-axis) for each of the 6, 18, 24 and 48 hr time points for acetaminophen does 150 mg/kg (shapes with white fill), 1500 mg/kg (shapes with black fill), and 2000 mg/kg (shapes with striped fill). Each data point represents the average for six animals.

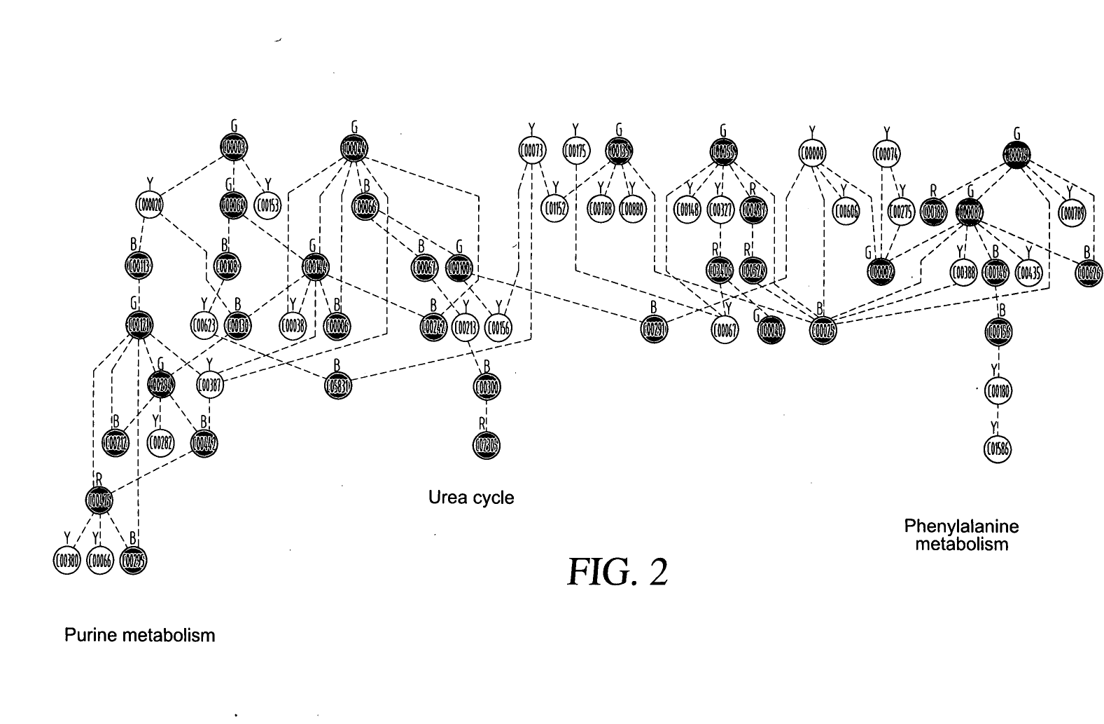

Mathematical models of the trends observed in the PCA plot were developed. Two trends were observed in the PCA plot. Trend 1 is a trend of increasing distance from control as the dose increases at any given time point. Trend 2 is a trend of change from one time point to the next that is in the same direction and of increasing magnitude as dose increases. Each of the relative response peaks in the liver biochemical profiling dataset was tested for adherence to the mathematical models to locate the subset of peaks that followed the observed trends. The peaks that adhered to the model were identified as the subset of most interest for further analysis of acetaminophen-induced liver changes and are listed in Table I. To further investigate the mode of action of acetaminophen, the known compounds in the identified subset were mapped to metabolic pathways. FIG. 2 is a diagram displaying that each of the identified compounds belongs to one of only three interconnected metabolic pathways, purine metabolism, urea cycle and phenylalanine metabolism. From this, it was hypothesized that the mode of action of acetaminophen toxicity affects these three adjacent pathways.

TABLE I
List of peaks located by trendFinder whose effect increases monotonically
with dose.
Compound Name

L-tyrosine
N-acetylneuraminic acid
agmatine
glutathione oxidized
peak03
peak05
peak12
peak13
peak24
phosphoethanolamine
2-ketobutyric acid
3,4-dihydroxyphenylalanine
L-histidine
L-ornithine
L-phenylalanine
N-acetyl-L-glutamate
argininosuccinic acid
beta-nicotinamide adenine dinucleotide
cytidine
peak10
peak11
peak16
phenylpyruvate
phosphocreatine
phosphocreatine
ribose
2,4-diamino-n-butyric Acid
2-ketobutyric acid
3,4-dihydroxyphenylalanine
GMP
L-cysteine
L-histidine
L-phenylalanine
L-proline
L-tyrosine
L-tyrosine
N-acetyl-L-glutamate
acetyl-L-carnitine
argininosuccinic acid
cystine
cytidine
gallic acid
guanosine
guanosine-5-triphosphate
hippuric acid
inosine
peak01
peak07
peak09
peak11
peak21
phosphoenolpyruvate
phosphoethanolamine
ribose
2-deoxyuridine
2-ketobutyric acid
3,4-dihydroxyphenylalanine
3-hydroxyanthranillic acid
GMP
L-histidine
L-methionine
L-phenylalanine
L-tyrosine
L-tyrosine
N-acetyl-L-glutamate
allantoin
beta-nicotinamide adenine dinucleotide
cystathionine
cystine
diaminopimelic acid
peak01
peak11
peak16
peak26
phosphoethanolamine
ribose

### Example 3

### Data Analysis Methods

### Locating a Subset of Most Interest that Corresponds to a Predicted Trend that is not Evident in a Dataset as a Whole

In this application of the invention, the targeted list of relative responses for peaks and peaks annotated as known compounds from Example 2 was analyzed to identify a subset that adhered to a predicted trend not evident in the dataset as a whole. The predicted trend of interest was a linear response across dose and a linear or quadratic response across time. Mathematical models representing the trend of interest were developed and each of the relative response peaks in the targeted list dataset was tested for adherence to the model. The subset of peaks that adhered to the designated trend is presented in Table II. Each of the compounds corresponding to the peaks in Table II is potentially implicated as having a role or being affected in acetaminophen induced liver toxicity.

TABLE II
Compounds associated with the mode of action of acetaminophen toxicity.
Compound name

2-deoxyuridine
2-ketobutyric acid
3′,5′-cyclic AMP
3,4-dihydroxyphenylalanine
GMP
L-asparagine
L-aspartate
L-citrulline
L-glutamine
L-histidine
L-lysine
L-ornithine
L-phenylalanine
L-serine
L-tyrosine
N-acetylneuraminic acid
argininosuccinic acid
beta-nicotinamide adenine dinucleotide
cystathionine
cysteic Acid
cytidine-3′-monophosphate
glucosamine-6-phosphate
glutathione oxidized
guanosine-5′-diphosphate
guanosine-5-triphosphate
lactic acid
malic acid
nicotinamide
orotidine
pantothenic acid
peak03
peak07
peak08
peak11
peak12
peak13
peak16
peak18
peak19
peak20
peak21
peak24
peak25
peak26
phosphocreatine
pipecolic acid
pyruvic acid
raffinose
ribose
sarcosine
taurine
xanthine

### Example 4

### Data Analysis Methods

### Locating a Subset of Most Interest that Corresponds to a Predicted Trend that is not Evident in a Dataset as a Whole

In this application of the invention, the mathematical model of Example 3 was modified to assume that the subjects would have shown zero difference from controls at time zero (this assumption is necessary because the subjects were not actually observed at time zero). As in Example 3, the predicted trend of interest was a linear response across dose and a linear or quadratic response across time, in addition to the assumption of a zero response at time zero. Mathematical models representing the foregoing trend of interest were developed. A subset of the relative response peaks in the targeted list dataset was extracted, namely those peaks that showed a significant difference from control at one or more doses and one or more timepoints. This subset was then tested for adherence to the mathematical model. The subset of peaks that adhered to the designated trend is presented in Table III. Each of the compounds corresponding to the peaks in Table III is potentially implicated as having a role or being affected in acetaminophen induced liver toxicity.

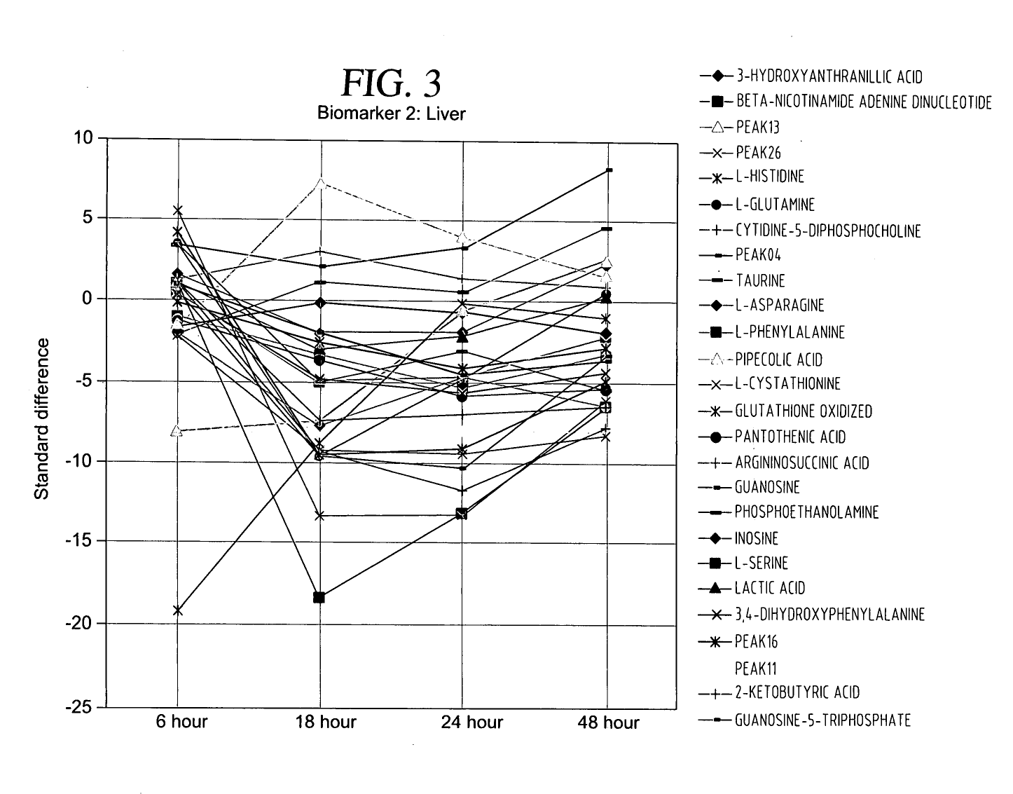

A graph of response relative to control in liver (standard difference; y-axis) versus time from acetaminophen (2000 mg/kg) dosing (x-axis) for each of the 24 peaks listed in Table III is shown in FIG. 3. The dataset corresponding to the graph in FIG. 3 is provided in Table listed below.

| Compound | 6 hour | 18 hour | 24 hour | 48 hour |
|---|---|---|---|---|
| 3-HYDROXYANTHRANILLIC | −1.60 | −0.17 | −0.64 | −1.81 |
| ACID | | | | |
| BETA-NICOTINAMIDE | 1.00 | −18.30 | −13.03 | −6.29 |
| ADENINE DINUCLEOTIDE | | | | |
| PEAK13 | −8.02 | −7.32 | −0.48 | 2.74 |
| PEAK26 | 5.52 | −13.21 | −13.13 | −6.01 |
| L-HISTIDINE | 4.20 | −9.40 | −9.03 | −4.73 |
| L-GLUTAMINE | 3.51 | −9.52 | −4.49 | 0.57 |
| CYTIDINE-5- | 1.06 | −9.20 | −11.62 | −7.69 |
| DIPHOSPHOCHOLINE | | | | |
| PEAK04 | 3.38 | 2.18 | 3.44 | 8.39 |
| TAURINE | 3.41 | −4.96 | −3.06 | −5.26 |
| L-ASPARAGINE | 1.57 | −2.14 | −1.84 | 2.42 |
| L-PHENYLALANINE | −1.03 | −3.21 | −5.31 | −3.17 |
| PIPECOLIC ACID | −1.69 | 7.34 | 4.08 | 1.74 |
| L-CYSTATHIONINE | −2.20 | −9.13 | −9.29 | −8.04 |
| GLUTATHIONE OXIDIZED | −19.11 | −8.64 | −0.16 | −0.90 |
| PANTOTHENIC ACID | −1.21 | −3.56 | −5.63 | −5.25 |
| ARGININOSUCCINIC ACID | 1.17 | 3.13 | 1.51 | 0.99 |
| GUANOSINE | 0.96 | −1.94 | −4.36 | −3.49 |
| PHOSPHOETHANOLAMINE | −2.07 | 1.08 | 0.57 | 4.72 |
| INOSINE | −2.05 | −7.66 | −4.53 | −6.30 |
| L-SERINE | 0.45 | −4.80 | −4.53 | −2.12 |
| LACTIC ACID | 1.15 | −2.90 | −2.02 | 0.38 |
| 3,4-DIHYDROXY- | 0.43 | −4.76 | −5.50 | −4.21 |
| PHENYLALANINE | | | | |
| PEAK16 | −0.15 | −2.50 | −4.03 | −2.73 |
| PEAK11 | −1.19 | −8.23 | −7.84 | −7.91 |
| 2-KETOBUTYRIC ACID | 1.08 | −7.30 | −6.90 | −6.31 |
| GUANOSINE-5-TRIPHOSPHATE | 0.51 | −9.47 | −10.17 | −3.28 |

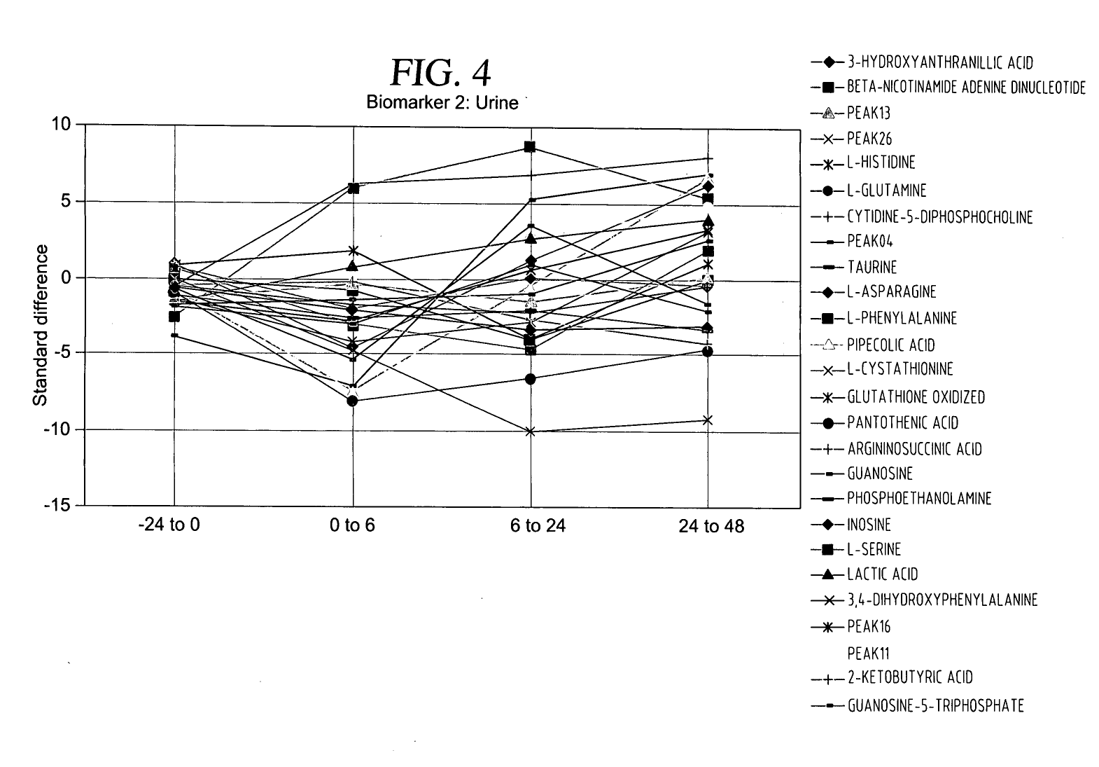

Similarly, a graph of response relative to control in urine (standard difference; y-axis) versus time from acetaminophen (1500 mg/kg) dosing (x-axis) for each of the 24 peaks listed in Table III is shown in FIG. 4. The dataset corresponding to the graph in FIG. 4 is provided in the Table listed below.

| Compound | −24 to 0 | 0 to 6 | 6 to 24 | 24 to 48 |
|---|---|---|---|---|
| 3-HYDROXYANTHRANILLIC | −0.639 | −2.074 | −3.277 | −3.040 |
| ACID | | | | |
| BETA-NICOTINAMIDE | −0.012 | −0.702 | −4.049 | 0.114 |
| ADENINE | | | | |
| DINUCLEOTIDE | | | | |
| PEAK13 | −1.405 | −0.349 | −1.470 | 0.251 |
| PEAK26 | −1.406 | −4.137 | −2.774 | 3.371 |
| L-HISTIDINE | 0.293 | −0.190 | −4.363 | −0.195 |
| L-GLUTAMINE | −3.731 | −6.887 | 3.283 | 5.042 |
| CYTIDINE-5- | −0.590 | 6.337 | 7.041 | 8.222 |
| DIPHOSPHOCHOLINE | | | | |
| PEAK04 | −1.740 | −1.423 | −0.831 | 2.838 |
| TAURINE | −0.731 | −5.364 | 3.648 | −1.488 |
| L-ASPARAGINE | 1.035 | −4.525 | 1.384 | 6.430 |
| L-PHENYLALANINE | 0.953 | −3.116 | −4.620 | 2.135 |
| PIPECOLIC ACID | −0.661 | −7.297 | −0.294 | 6.938 |
| L-CYSTATHIONINE | −0.044 | −4.735 | −10.037 | −9.221 |
| GLUTATHIONE OXIDIZED | 0.000 | 0.000 | 0.000 | 0.000 |
| PANTOTHENIC ACID | −0.947 | −7.939 | −6.507 | −4.507 |
| ARGININOSUCCINIC ACID | −0.401 | −0.255 | −2.754 | −4.186 |
| GUANOSINE | −3.832 | −7.091 | 5.388 | 7.177 |
| PHOSPHOETHANOLAMINE | −1.142 | −2.631 | −2.025 | −3.210 |
| INOSINE | 0.647 | −1.945 | 0.224 | −0.293 |
| L-SERINE | −2.582 | 6.056 | 8.840 | 5.537 |
| LACTIC ACID | −1.397 | 0.818 | 2.909 | 4.206 |
| 3,4- | −1.531 | −2.719 | 0.680 | 3.560 |
| DIHYDROXY- | | | | |
| PHENYLALANINE | | | | |
| PEAK16 | 0.862 | 1.828 | −3.984 | 1.223 |
| PEAK11 | −0.169 | 0.299 | 2.873 | 5.043 |
| 2-KETOBUTYRIC ACID | −0.500 | −1.666 | −2.145 | −0.294 |
| GUANOSINE-5-TRIPHOSPHATE | −1.801 | −3.008 | 1.040 | −1.969 |

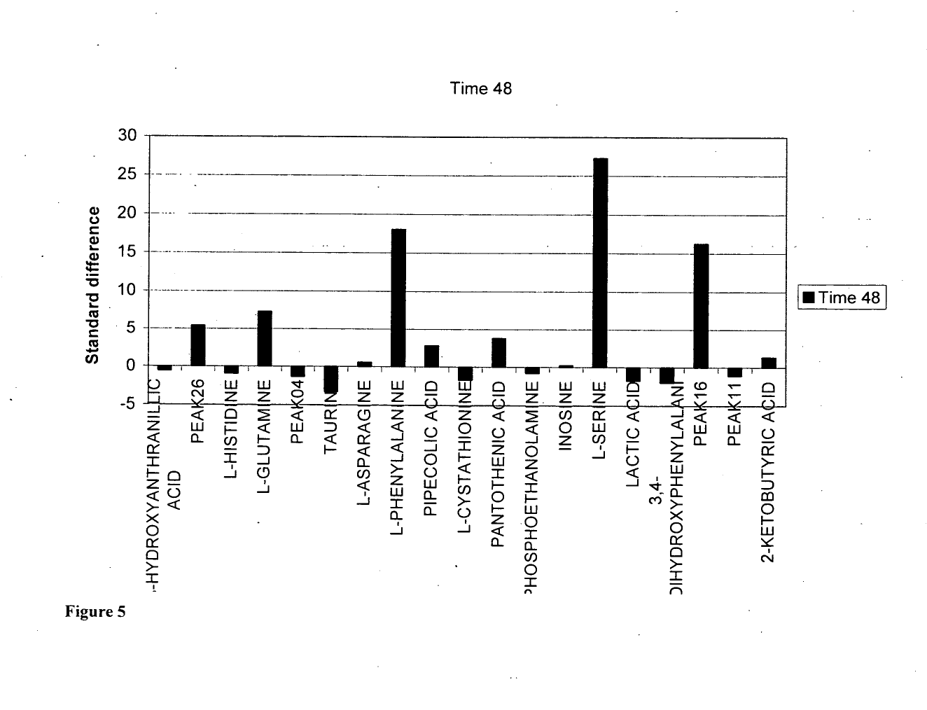

FIG. 5 is a graph of response relative to control in serum (standard difference; y-axis) at 48 hours after acetaminophen (1500 mg/kg) dosing for each of the peaks listed in Table III (x-axis). Measurement of relative response in serum or urine for any combination of one or more of the response peaks listed in Table III may be useful as an early biomarker of liver damage and/or disease. The combinations of response peaks having the highest explanatory power of the observed trend and statistically significant changes among dose/time groups in the target tissue are the preferred combinations for biomarker use.

TABLE III
A biomarker suite of acetaminophen induced liver toxicity.
Compound

3-hydroxyanthranillic acid
beta-nicotinamide adenine dinucleotide
peak13
peak26
l-histidine
l-glutamine
cytidine-5-diphosphocholine
peak04
taurine
l-asparagine
l-phenylalanine
pipecolic acid
l-cystathionine
glutathione oxidized
pantothenic acid
argininosuccinic acid
guanosine
phosphoethanolamine
inosine
l-serine
lactic acid
3,4-dihydroxyphenylalanine
peak16
peak11
2-ketobutyric acid
guanosine-5-triphosphate

### Example 5

### Data Analysis Methods

### Locating a Subset as a Putative Biomarker in a Target Tissue

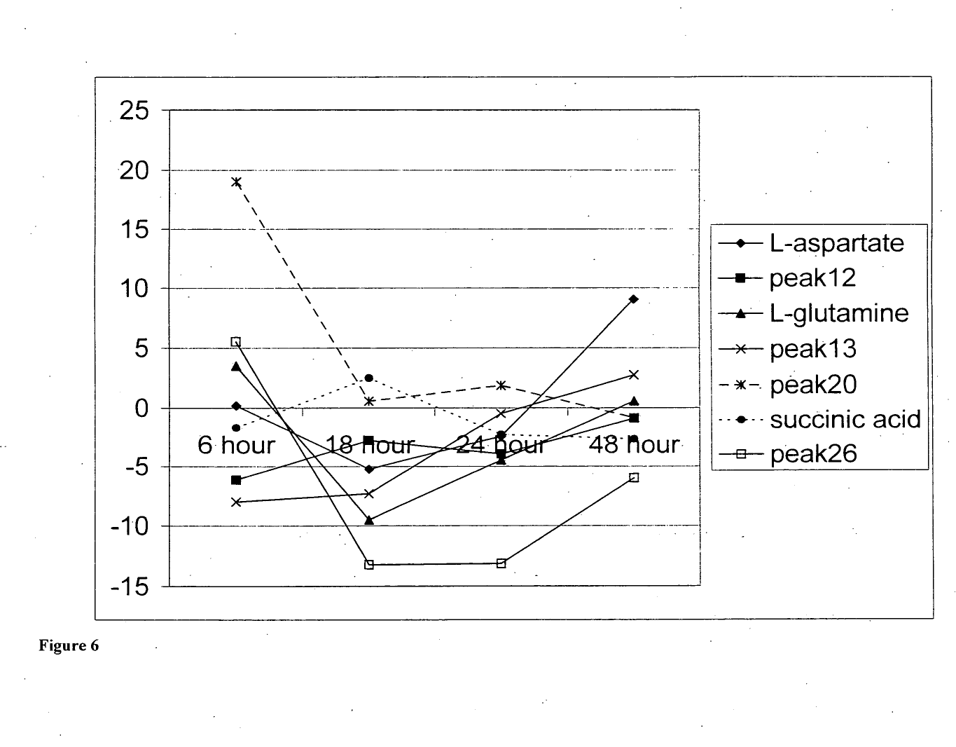

The subset of peaks identified for rat liver in Example 2 as being of most interest for investigation into acetaminophen induced liver damage (Table I) was further analyzed for an ability to act as a putative biomarker of acetaminophen toxicity in a target tissue such as serum or urine. In this application, stepwise discriminant analysis (SDA) was performed on the subset of relative response peak data points identified in liver to extract a relatively small group of the data points with highest explanatory power of the observed PCA plot trend and statistically significant changes among dose/time groups. The forgoing analysis resulted in the small group of 7 peaks/compounds that are listed in Table IV. Compounds corresponding to the 7 peaks listed in Table IV are hypothesized to be associated with acetaminophen liver toxicity. A graph of response relative to control (standard difference; y-axis) versus time from dosing (x-axis) for each of the 7 peaks in liver tissue is shown in FIG. 6.

TABLE IV
Putative biomarker suite.
Compound Name

aspartic acid
Peak12
glutamine
Peak13
Peak20
succinic acid
Peak26

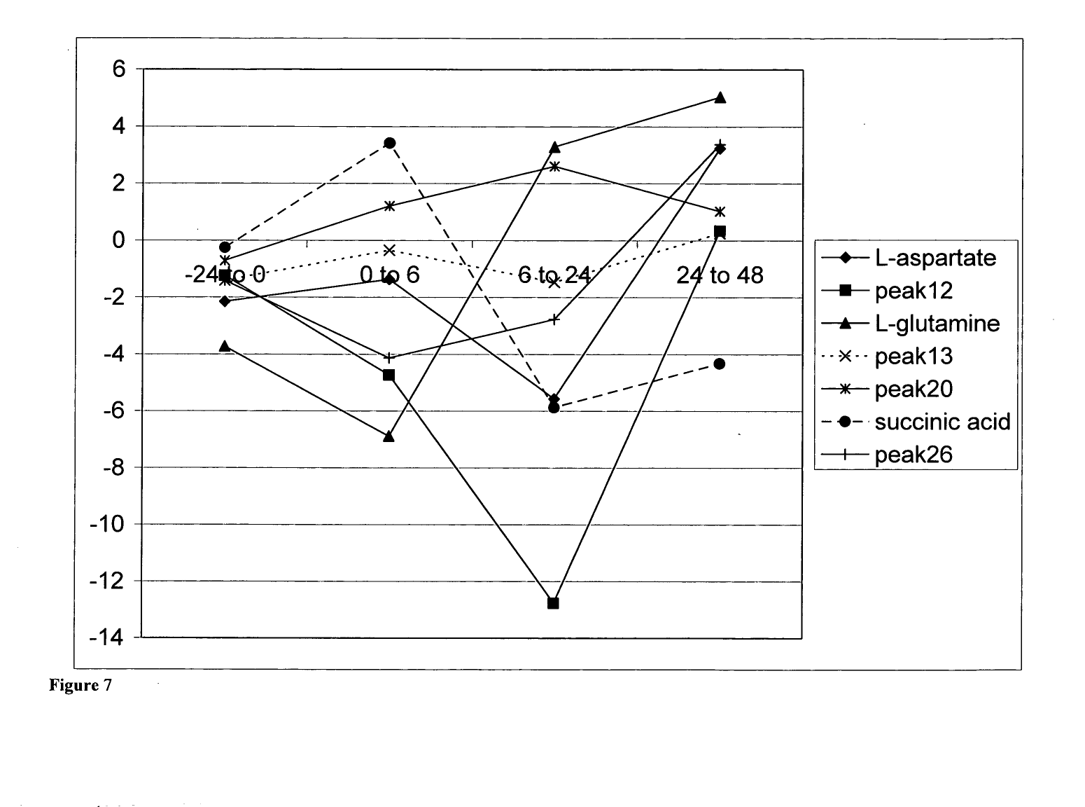

To determine whether the responses of the small group of 7 biochemical entities extracted from the rat liver data have potential for use as a biomarker of liver toxicity in a target tissue, such as serum or urine, the relative response of the 7 entities was analyzed in the biochemical profiling data for the rat serum and urine collected along with the liver data in the study described in Example 1. Upon analysis, 6 of the 7 liver entities were both present in serum and urine, and the responses of the 6 entities present showed a significant variation from control (baseline) in both the urine and serum samples. The 6 entities are aspartic acid, Peak12, glutamine, Peak20, succinic acid, and Peak26. Peak13 was not observed in serum and the response for Peak13 in urine did not vary significantly from control (FIG. 7).

Specifically, the forgoing 6 peak responses met the following criteria established for a potential biomarker: i) the peak responses were among those rated most highly by the SDA, ii) each of the peak responses varied significantly from baseline in the rat liver subset, and iii) because the liver subset was not the target tissue dataset, each of the peak responses in the subset was detectable in the target tissue (i.e. serum and urine in the present case) and varied significantly from the baseline in the target tissue datasets.

Having met the criteria for a potential biomarker of liver toxicity in serum and urine, the small group of 6 data points was subjected to parametric discriminant analysis to obtain a discrimination score that measures how well the small group distinguishes between the experimental groups of the target dataset, wherein a high discrimination score indicates the small group as a putative biomarker of the trend of interest in the target tissue. A discrimination score of 100% led to the hypothesis that the relative response of compounds corresponding to the 6 peaks present in serum and urine is potentially useful as a biomarker for acetaminophen hepatotoxicity, as the responses are associated with the toxic effects of acetaminophen on liver but detectable in the target tissues, serum and urine.

### Example 6

### Data Analysis Methods

### Locating a Subset of Gene Expression Data of Most Interest that Corresponds to an Observed Trend

A list of relative responses for annotated genes is produced for the liver samples subjected to gene expression profiling via microarray analysis as described in Example 1. The resulting dataset consists of probes (subsets of genes) and corresponding expression levels. PCA is performed on the gene expression data. Similar to that described in Example 2, mathematical models of the trends observed in the PCA plot can be developed and each of the relative response data points in the liver gene expression profiling dataset tested for adherence to the mathematical models to locate the subset of genes that follows the observed trends. The genes that adhere to the model are identified as the subset of most interest for further analysis of acetaminophen-induced liver changes. To further investigate the mode of action of acetaminophen, the genes in the identified subset are mapped to metabolic pathways. In this manner, hypotheses can be generated about the mode of action of acetaminophen toxicity.

### Example 7

### Metrics

### Evaluation of Candidate Biomarker Suite II

In this application of the invention, the performance of the candidate biomarker suite listed in Table III (Candidate Biomarker Suite II) was objectively evaluated using the following statistical and biological metrics: Accuracy, Cross-validation Accuracy, Confusion metric, TRENDFINDER Score, and NODEWALKER results. The methods and resulting statistical scores and biological relevance of Biomarker Suite II are displayed below.

### Accuracy: Score=97.5% Correct

The Accuracy is defined as the percentage of correct classification achieved on the original data set. This method generally over-estimates the accuracy of the classification approach applied to a new data point from the same population, since the accuracy is measured on the same dataset from which the discriminant rule was derived.

### Cross-Validation Accuracy: Score=67.5% Correct

The Cross-validation Accuracy is a more objective assessment of accuracy, which is defined as the percentage of correct classifications using different training and testing data sets. The leave-on-out approach was used to calculate the cross-validation accuracy. In this approach the training and testing data was formed by dividing the dataset into training and testing sets by taking one instance as a test sample and the remaining (N−1) samples as training examples. The training data was used to induce the discrimination function and the test instance used to calculate the classification accuracy. The method provides an unbiased estimate of the accuracy of the classification rule applied to a new data point from the same population.

### Confusion Metric: Score=0.17% Confusion

The confusion score was calculated as the average distance between an observation's true class and the class into which it was classified (see “Accuracy” above). This average was calculated over all observations in the original dataset. As such, it provides additional information to the accuracy score, because the confusion score will increase as the classification moves further away from the true class. In this case, the distance metric used was Manhattan (a.k.a. “city-block”) distance. That is, any two adjacent classes are separated by a distance of 1.

### Formally,

let C be the classification function
let Sᵢⱼ be the jth subject in the ith treatment group.
Let n be the number of subjects
Let C(Sᵢⱼ)=cᵢⱼ
Let ∥x,y∥ denote the Manhattan distance between x and y.

Let dij=∥i,cᵢⱼ∥

### Then the Confusion Score=1/n ΣΣdij

### TRENDFINDER Score: Score=85%

The TRENDFINDER score was calculated from the p-values assigned to the peaks in the algorithms described in Examples 2 and 3. The score was calculated as follows:
Each peak was assigned a p-value for dose, time, time², and time³.
If the p-value for time³ was <0.01, it was subtracted from the others, to penalize for a cubic trend in time.
The average p-value over all peaks was calculated.
The TRENDFINDER Score=100(1−average p-value).
   This score will be larger for compound sets that more closely match the trend.

### Biological Relevance: NodeWalker Results:

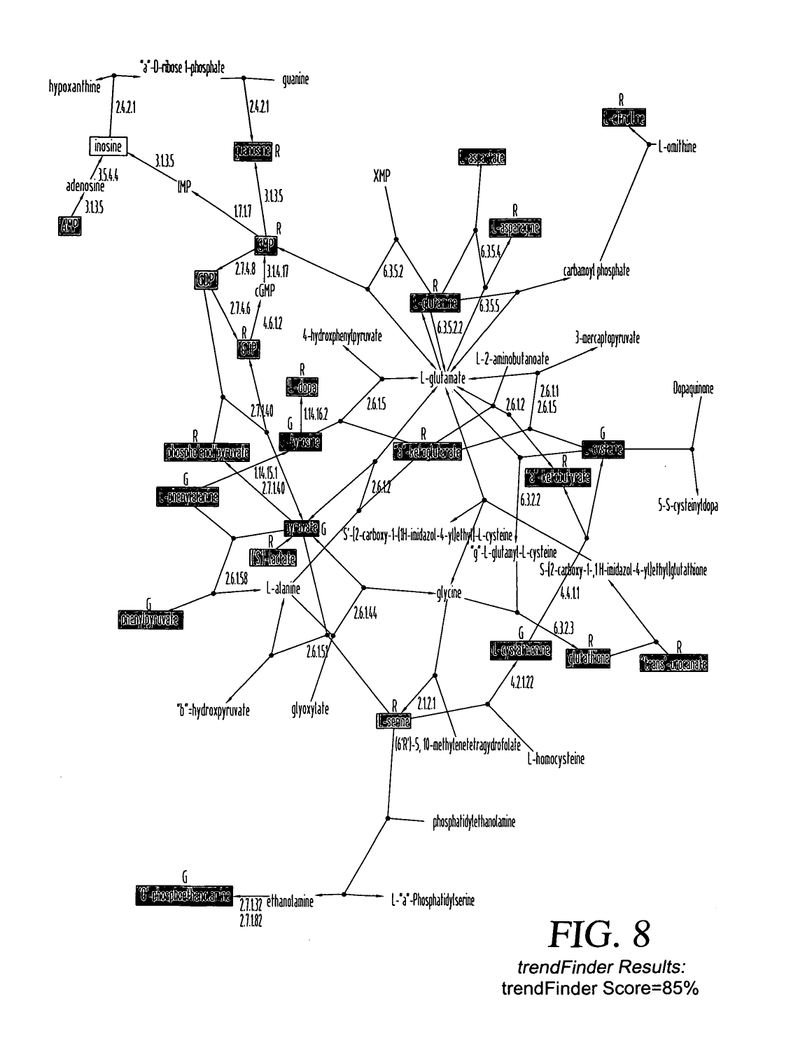

Suite II contains 26 peaks, 21 of which were mapped to a KEGG identifier. 12 of these identified compounds map to a single subcluster, with 53 total compound nodes. The remainder of the identified compounds were mapped to disconnected singletons. The sub-network shown in FIG. 8 comprises parts of purine and pyrimidine metabolism, alanine and aspartate metabolism, and a branch leading to glycerolipid metabolism at the bottom of the figure.

### Example 8

### Data Analysis Methods

### Locating a Subset of Most Interest to a Clinical Endpoint that Corresponds to a Predicted Trend that is not Evident in a Dataset as a Whole

In this application of the invention, the mathematical model of Example 4 was used. A subset of the relative response peaks in the targeted list dataset was extracted, namely those peaks that showed a significant difference from control at one or more doses and one or more timepoints. This subset was then tested for adherence to the mathematical model. The subset of peaks that adhered to the designated trend was then further screened by stepwise discriminant analysis to choose the subset that best distinguishes between classes of the clinical endpoint, which is a pathologist's rating of the liver tissue slide. The ratings are on the following scale: 0=normal tissue; 1=Minimal Inflammation; 2=Minimal Necrosis; 3=Dead. Each of the peaks corresponding to the peaks in Table V is potentially implicated as having a role in or being affected by acetaminophen induced liver damage.

TABLE V
A biomarker suite of acetaminophen induced liver damage.
Component (mass followed by retention time)

n_100.1_1.30
n_119.0_1.13
n_128.0_1.03
n_161.1_1.10
n_162.1_1.10
n_263.1_1.10
n_267.1_1.10
n_335.1_1.27
n_402.2_1.10
n_521.2_1.10
n_656.3_0.99
n_683.4_1.10
p_158.1_1.72
p_202.2_1.58
p_523.3_1.06

### Example 9

### Acetaminophen-Induced Liver Toxicity Study Design #2

### GEP Data Acquisition and Analysis

### Study Design:

An extended study on APAP-induced liver toxicity was conducted in Fisher F344 rats. Rats were treated with a single oral dose of APAP at 0, 150, 450 and 2000 mg/kg. Animals were sacrificed at 0, 6, 18, 24 and 48 hours, and liver samples were obtained for the analysis of biochemical profiles, gene expression, histopathology and histomorphometric profiling. Serum samples were also obtained at the above times for routine clinical chemistry and biochemical profiling. Urine was collected from the latest group of rats for biochemical profiling over the following time periods: 24 hours pre-dose, 0-6 hours, 6-24 hours and 24-48 hours. Preliminary analysis of the clinical chemistry revealed no changes in liver enzymes in the low dose group, modest increases in the mid dose group at 48 hours only, and marked elevations in the high dose group from 18 hours onwards.

### Data Acquisition:

The resulting 90 liver samples were analyzed using GEP and BCP at all doses and times. Measurements were made of 15,923 probes from GEP (using the Affymetrix RAE203a genechip) and of 646 high-confidence metabolite peaks from BCP (using liquid chromatography and the Mariner™ Biospectrometry™ Workstation, an ESI-TOF mass spectrometer). GEP was performed more specifically as described below. The following major steps outline standard GEP protocols used for GeneChip expression analysis: (Please note: during the laboratory procedure described here, biotin-labeled RNA hybridized to the probe array are referred to as the “target”.)

1. Target Preparation (the so-called “T7 protocol”)
For eukaryotic samples, double-stranded cDNA was synthesized from total RNA or purified poly-A messenger RNA isolated from tissue or cells. An in vitro transcription (IVT) reaction was done to produce biotin-labeled cRNA from the cDNA. The cRNA was fragmented before hybridization.

### 2. Target Hybridization

A hybridization cocktail was prepared, including the fragmented target, probe array controls, BSA, and herring sperm DNA. It was then hybridized to the probe array during a 16-hour incubation.

### 3. Experiment and Fluidics Station Setup

Specific experimental information was defined using Affymetrix® GeneChip Operating Software. The probe array type, barcode, chip lot number, sample description, and comments were entered and saved with a unique experiment name. The fluidics station was then prepared for use by priming with the appropriate buffers.

### 4. Probe Array Washing and Staining

Immediately following hybridization, the probe array underwent an automated washing and staining protocol on the fluidics station.

### 5. Probe Array Scan

Once the probe array had been hybridized, washed, and stained, it was scanned. Each workstation running Affymetrix® GeneChip Operating Software can control one scanner. The software defines the probe cells and computed an intensity for each cell. Each complete probe array image was stored in a separate data file identified by the experiment name and was saved with a data image file (.dat) extension.

### 6. Data Analysis

Data was analyzed using the Microarray Suite Expression Analysis window. The .dat image was analyzed for probe intensities; results were reported in tabular and graphical formats.

### Data Analysis

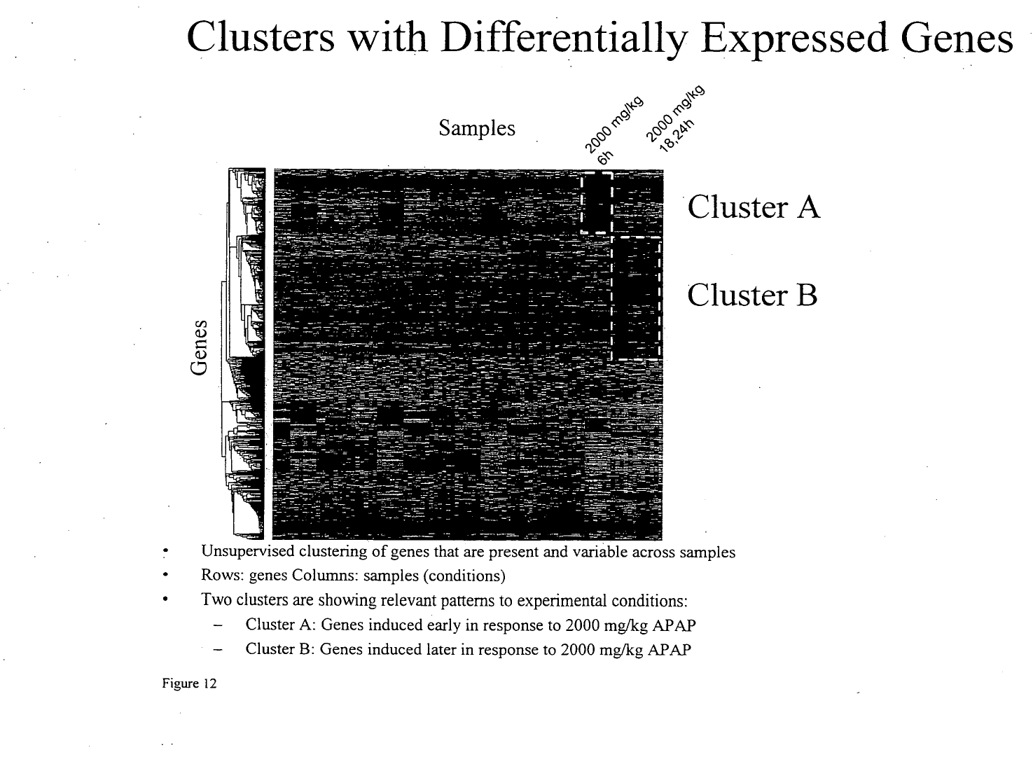

The GEP data were analyzed by gene filtering and clustering according to the following procedure. Results of the analysis are displayed in (FIG. 12).

1. Genes having flat patterns were filtered out using Coefficient of Variation (CV): 0.2<CV<1000. CV is the ratio of the standard deviation over the mean of a gene's expression values across all samples. The more variable a gene is across samples, the larger the ratio is.
2. Absent genes were filtered out: in the foregoing experiment a gene was required to be called “Present” in more than 20% of the arrays in the array list file.
3. Before clustering, the expression values for a gene across all samples were standardized (linearly scaled) to have mean 0 and standard deviation 1, and these standardized values were used to calculate correlations between genes and samples and to serve as the basis for merging nodes.
4. The samples were clustered using a hierarchical agglomerative clustering algorithm with average linkage. Clusters were formed at a correlation cutoff of 0.2.

### Example 10

### Acetaminophen-Induced Liver Toxicity

### Rat Primary Hepatocytes

### Study Design:

An acetaminophen-induced liver toxicity study was performed as follows. Experimental groups of rat primary hepatocytes were exposed to 0.2 mM, 1.0 mM, 1.2 mM, 10 mM, and 30 mM APAP for 4 or 18 hours, respectively. Control groups were not exposed to APAP. Cell viability was assessed by ATP content. BCP analysis was carried out by LC-MS, as described in Example 1 (data not shown) and the experimental groups compared to control groups by various statistical measures.

The following biomolecules were identified as hepatocyte markers of glutathione turnover: Glutathione, glutamine, 2-ketobutarate, cystathionine, taurine, and serine.

In addition, the data was analyzed to determine biomolecules that were significantly changed in rat liver, serum, urine, and primary hepatocytes (summarized in Table VI, below).

TABLE VI
Biochemicals significantly changed in rat liver, serum, urine, and
primary hepatocytes

| | Biological Sample | | |
|---|---|---|---|
| | | | Primary |
| Biochemicals | Serum | Urine | Hepatocytes |
| Phenylalanine | x | | x |
| Tyrosine | | x | |
| Cystathionine | x | x | x |
| Serine | x | x | x |
| Lysine | x | x | x |
| Pipecolate | x | x | |
| Pantothenate | | x | x |
| Citrulline | x | | |
| Ribose | | x | |

(x = significantly changed).

### Example 11

### Acetaminophen-Induced Liver Toxicity

### Human Subjects

### Study Design:

An acetaminophen-induced liver toxicity study was performed as follows. Six healthy human volunteers received a standardized soy shake diet for 72 hours. Each volunteer received a single oral dose of 4 g APAP (˜50 mg/kg). Serum and urine were sampled at 0, 6, 18, 24, 48, 72 and 96 hours (urine collected between these times). BCP analysis was carried out as described in Example 1. Data was analyzed to determine biomolecules that were significantly changed in both rat and human serum and urine (summarized in Table VII, below).

TABLE VII
Biochemicals significantly changed by APAP in both rat and human
biological samples.
Correlations Between Rat and Human Biofluids

| Biochemicals | Serum | Urine |
|---|---|---|
| Phenylalanine | X | |
| Glutamate | X | X |
| Glutamine | X | |
| Serine | | X |
| Lysine | | X |
| Pipecolate | X | |
| 5-Hyroxytryptophan | | X |
| Citrulline | X | |
| Ornithine | X | |
| Creatinine | X | |
| Homoserine | X | |
| 2-Oxoglutarate | | X |
| Guanosine | X | |
| Hypoxanthine | X | |
| Cytosine | | X |

What is claimed is:

1. A method of detecting liver injury in a subject, comprising:
analyzing a biological sample from a subject using one or more selected from one or more biomarkers listed in Table III, and combinations thereof; and
comparing the relative level(s) of the one or more biomarkers in the sample to levels of the one or more biomarkers from a control sample; and
determining whether or not the subject has liver damage.

2. The method of claim 1, wherein the sample is obtained from liver tissue, serum, or urine.

3. The method of claim 2, wherein the sample is obtained from urine.

4. A method for monitoring liver injury in a subject comprising the steps of:
(a) measuring at least one biomarker in a sample from the subject, wherein the biomarker is selected from the group consisting of 3-hydroxyanthranillic acid, beta-nicotinamide adenine dinucleotide, 1-histidine, 1-glutamine, cytidine-5-diphosphocholine, taurine, 1-asparagine, 1-phenylalanine, pipecolic acid, 1-cystathionine, glutathione oxidized, pantothenic acid, argininosuccinic acid, guanosine, phosphoethanolamine, inosine, I-serine, lactic acid, 3,4-dihydroxyphenylalanine, 2-ketobutyric acid, and guanosine-5-triphosphate and;
(b) testing the measurement to determine its adherence to a mathematical model of liver injury.

5. The method of claim 4, wherein the at least one biomarker includes aspartic acid, glutamine, and succinic acid.

6. The method of claim 4, wherein the at least one biomarker is selected from the group consisting of aspartic acid, glutamine, and succinic acid.

7. The method of claim 4, wherein the testing is done by determining whether the biomarker is significantly elevated.

8. The method of claim 4, wherein the biomarker is measured at more than one time point.

9. The method of claim 8, wherein the testing is done by determining whether the measurement increases linearly over time.

10. The method of claim 8, wherein the testing is done by determining whether the measurement increases quadratically over time.

11. The method of claim 4, wherein the samples are selected from the group consisting of liver tissue, serum samples, and urine samples.

12. A method for monitoring liver injury caused by a hepatotoxicant in a subject comprising the steps:
(a) measuring at least one biomarker in a sample from the subject, wherein the at least one biomarker is selected from the group consisting of 3-hydroxyanthranillic acid, beta-nicotinamide adenine dinucleotide, 1-histidine, 1-glutamine, cytidine-5-diphosphocholine, taurine, 1-asparagine, 1-phenylalanine, pipecolic acid, 1-cystathionine, glutathione oxidized, pantothenic acid, argininosuccinic acid, guanosine, phosphoethanolamine, inosine, I-serine, lactic acid, 3,4-dihydroxyphenylalanine, 2-ketobutyric acid, and guanosine-5-triphosphate;
(b) obtaining samples from a plurality of subjects exposed to known amounts of the hepatotoxicant over a period of time by
(i) classifying samples taken from a plurality of subjects in different groups according to dose of hepatotoxicant and time;
(ii) measuring the at least one biomarker of (a) in each of the samples of (b); and
(c) testing the measurement to determine its adherence to a mathematical model of liver injury, wherein the mathematical model of liver injury is developed from the measurements of (b).

13. The method of claim 12, wherein the at least one biomarker includes aspartic acid, glutamine, and succinic acid.

14. The method of claim 12, wherein the at least one biomarker is selected from the group consisting of aspartic acid, glutamine, and succinic acid.

15. The method of claim 12, wherein the biomarker is measured at more than one time point.

16. The method of claim 15, wherein the correlating is done by determining whether the measurement increases linearly over time.

17. The method of claim 16, wherein the correlating is done by determining whether the measurement increases quadratically over time.

18. The method of claim 12, wherein the samples are selected from the group consisting of liver tissue samples, serum samples, and urine samples.
